(19) United States
(12) Patent Application Publication
(10) Pub. No.: US 2003/0167075 A1
(43) Pub. Date: Sep. 4, 2003

(54) Automated external defibrillator (AED) system

(76) Inventor: Randall Fincke, Concord, MA (US)

Correspondence Address: Pandiscio & Pandiscio, 470 Totten Pond Road, Waltham, MA, 02154, US

(21) Appl. No.: 10/232,645

(22) Filed: Aug. 30, 2002

Related U.S. Application Data
(60) Provisional application No. 60/316,034, filed on Aug. 31, 2001.
(60) Provisional application No. 60/379,467, filed on May 10, 2002.

(51) Int. Cl. A61N 1/39

(52) U.S. Cl. 607/8

(57) ABSTRACT

An automated external defibrillator (AED) system comprising a defibrillator and its associated electrodes. The defibrillator is compact, rugged, lightweight, inexpensive, easy to use, water-resistant and electronically efficient, wherein the defibrillator is in the form of a unit (or box) with a lid and a body. The body houses the electronics associated with the defibrillator and the lid houses the electrodes. Furthermore, the defibrillator uses a unique hardware design that utilizes a stacked, switched capacitor design to generate bi-phasic waveforms, thereby providing for a compact defibrillator unit. The unit further comprises a liquid crystal display (LCD) that displays pertinent information such as electrocardiogram (ECG) graphs, and a voice-based system that helps guide the user through the defibrillation process. The electrodes of the defibrillator are sealed in a tray that is attached to the interior of the lid.

### REFERENCE TO PENDING PRIOR PATENT APPLICATIONS

This patent application claims benefit of:

(1) pending prior U.S. Provisional Patent Application Serial No. 60/316,034, filed Aug. 31, 2001 by Randall Fincke for DEFIBRILLATOR AED UNIT (Attorney's Docket No. PA-3003869); and

(2) pending prior U.S. Provisional Patent Application Serial No. 60/379,467, filed May 10, 2002 by Randall Fincke for AUTOMATED EXTERNAL DEFIBRILLATOR (AED) SYSTEM (Attorney's Docket No. ACCESS-2 PROV).

The two foregoing patent applications (including any and all appendices thereto) are hereby incorporated herein by reference.

### FIELD OF THE INVENTION

This invention relates to defibrillators in general, and more particularly to automated external defibrillators (AED's).

### BACKGROUND OF THE INVENTION

The heart pumps blood through the body using coordinated heart muscle contractions. Ventricular fibrillation is a chaotic heart rhythm that causes an uncoordinated quivering of the heart muscle. The lack of coordinated heart muscle contractions results in the loss of blood flow to the brain and other organs. This is sometimes referred to as cardiac arrest. While cardiopulmonary resuscitation (CPR) can sustain a patient in cardiac arrest for a short time, only defibrillation can restore a normal heart rhythm. Without defibrillation, the victim will die.

In certain cases, ventricular tachycardia can also cause cardiac arrest. More particularly, ventricular tachycardia is a very rapid heart rhythm which can also cause a loss of blood flow. Like ventricular fibrillation, the only effective treatment for pulseless ventricular tachycardia is defibrillation.

Defibrillators are commonly used to treat ventricular fibrillation and ventricular tachycardia. Defibrillators are electronic devices that apply an electric pulse to stop the chaotic fibrillation of the heart and restore the normal heart rhythm. There are a variety of different types of defibrillators, but most can be classified into two categories: internal (sometimes referred to as implanted) defibrillators and external defibrillators.

Internal (or implanted) defibrillators are provided for people whose heart is at considerable risk of fibrillation at some point in the near future. Therefore, physicians predict the need for electro-therapy in a patient and implant, via surgery, a defibrillator. A mechanism is provided in the implanted defibrillator for monitoring heart rhythms and, when the detected rhythm suggests fibrillation, the implanted defibrillator generates an electric pulse that stops fibrillation and restores the normal heart rhythm. One major advantage of internal defibrillators is that they can be customized for each and every patient, taking into account a variety of different parameters associated with that specific patient.

External defibrillators, as the name suggests, are applied externally to the patient. These defibrillators are typically used in hospitals, emergency rooms, offices, airplanes, etc., where electro-therapy might be required on short notice. In such places, there is a need for a defibrillator (such as an external defibrillator) that can be applied quickly and work dynamically with the varying parameters associated with different patients. External defibrillators provide these features, able to be applied quickly in an emergency situation and working effectively with many different patients so as to stop fibrillation and restore the normal heart rhythm.

As noted above, external defibrillators are applied externally to the patient and deliver an electric pulse that propagates to the heart. In other words, the pulse generated by the defibrillator passes through the skin of the patient, travels through the tissue of the thorax and finally reaches the heart. There is typically a significant impedance associated with this propagation pathway. Thus, the generated pulse needs to have considerable voltage in order to overcome the impedance associated with the intervening tissue. At the same time, the pulse must have sufficient current to achieve the therapeutic effect. This need for considerable energy (high voltage and sufficient current) generally complicates hardware design and typically makes prior art external defibrillator systems large, heavy and expensive.

Another problem associated with external defibrillators is the difficulty in providing an appropriate electric pulse to the heart. More particularly, as noted above, in external defibrillators, the defibrillator pulse must overcome the impedance of the tissue lying between the defibrillator and the heart. However, it has been found that the impedance of the intervening tissue varies significantly from patient to patient. Thus, a measurement should be made to determine the specific impedance associated with each particular patient. The amount of shock or pulse voltage needed for effective defibrillation is directly related to this measured impedance: the greater the impedance, the greater the voltage that is required in order to overcome the impedance associated with the tissue.

Some prior art defibrillators precisely measure the impedance associated with each patient and then, based on this measured impedance, charge to a specific, pre-selected target discharge voltage before firing the electric pulse. However, this approach tends to increase the size, cost, complexity and sophistication of the device. In addition, this arrangement cannot be used with paddle-type defibrillators. Other prior art defibrillators use fixed voltages and do not attempt to regulate current. Thus, they allow the current to vary significantly with patient impedance. However, this latter design can result in the generation of excessively high currents for low thoracic impedance patients, which can cause burning or other injury to the patient, including cardiac stunning and reduced efficacy; and this latter design can result in the generation of inadequately low currents for high thoracic impedance patients, which can result in ineffective defibrillation.

Automated external defibrillators (AED's) are also known in the prior art. AED's are designed to automatically analyze the victim's heart rhythm and, if it is found to be in fibrillation, deliver an appropriate electric pulse (or “shock”) to the heart so as to restore the normal heart rhythm. Due to their more automated nature, AED's can be successfully used in a wider range of locations (e.g., airplanes) by a wider range of first-responder personnel (e.g., flight attendants), thereby significantly reducing the response time for patients in cardiac arrest and thus significantly increasing their chance for survival. In this respect it should be appreciated that if defibrillation is applied within 5 minutes of the onset of a cardiac arrest, there is an approximately 50% chance for survival. However, survival rates drop by approximately 7-10% with every minute that passes after that. In essence, after approximately 10 minutes, there is relatively little chance for survival. Unfortunately, however, for many emergency response operations, the relatively high cost of conventional AED's can be hard to justify, thus limiting the availability of AED's for the general public.

None of the prior art defibrillators described above provide for a unit that is compact, rugged, lightweight, inexpensive, easy to use, water-resistant and electronically efficient. Whatever the precise merits, features and advantages of the above-described prior art defibrillators, none of them achieves or fulfills the purposes of the present invention.

### SUMMARY OF THE INVENTION

The present invention provides a novel automated external defibrillator (AED) system that comprises an automated external defibrillator (AED) and a pair of associated electrodes.

More particularly, in one form of the invention there is provided a defibrillator for applying a therapeutic shock pulse to a patient, the defibrillator being adapted to: (1) measure the thoracic impedance of the patient; and (2) provide a bi-phasic shock pulse to the patient, the bi-phasic shock pulse: (i) being characterized by a tilt which is less than the time constant of a 100 μF capacitance; and (ii) having a peak current limited in accordance with the measured impedance of the patient.

In another aspect of the invention, there is provided a defibrillator for applying a therapeutic shock pulse to a patient, the defibrillator being adapted to: (1) measure the thoracic impedance of the patient; and (2) provide a bi-phasic shock pulse to the patient, the bi-phasic shock pulse: (i) being characterized by a tilt which is less than the time constant of a 100 μF capacitance; and (ii) having a shock voltage selected in accordance with the measured impedance of the patient, wherein the shock voltage is generated by apparatus charged to a fixed charge voltage.

In another aspect of the invention, there is provided a defibrillator for applying a therapeutic shock pulse to a patient, the defibrillator being adapted to: (1) measure the thoracic impedance of the patient; and (2) provide a bi-phasic shock pulse to the patient, the bi-phasic shock pulse: (i) being characterized by a tilt which is less than the time constant of a 100 μF capacitance; (ii) having a peak current limited in accordance with the measured impedance of the patient; and (iii) having a shock voltage selected in accordance with the measured impedance of the patient, wherein the shock voltage is generated by apparatus charged to a fixed charge voltage.

In another aspect of the invention, there is provided a defibrillator for applying a therapeutic shock pulse to a patient, the defibrillator being adapted to: (1) measure the thoracic impedance of the patient; and (2) provide a bi-phasic shock pulse to the patient, the bi-phasic shock pulse: (i) being characterized by a tilt which varies in accordance with the measured impedance of the patient; and (ii) being characterized by a tilt which is less than the time constant of a 100 μF capacitor.

In another aspect of the invention, there is provided a defibrillator for applying a therapeutic shock pulse to a patient, the defibrillator being adapted to: (1) measure the thoracic impedance of the patient; and (2) provide a bi-phasic shock pulse to the patient, the bi-phasic shock pulse: (i) being characterized by a tilt which is less than the time constant of a 100 μF capacitance; (ii) having a time duration selected in accordance with the measured impedance of the patient; and (iii) having a peak current limited in accordance with the measured impedance of the patient.

In another aspect of the invention, there is provided a defibrillator for applying a therapeutic pulse to a patient, the defibrillator being adapted to:

(1) measure the thoracic impedance of the patient; and

(2) provide a bi-phasic shock pulse to the patient, the bi-phasic shock pulse: (i) being characterized by an increased average current in accordance with the measured impedance of the patient.

In another aspect of the invention, there is provided a defibrillator for applying a therapeutic shock pulse to a patient, the defibrillator comprising: a body enclosing hardware for generating the shock pulse; and a lid for covering all of the user accessible components of the body.

In another aspect of the invention, there is provided a defibrillator for applying a therapeutic shock pulse to a patient, the defibrillator comprising: a body enclosing hardware for generating the shock pulse; and a lid for covering at least a portion of the body, the lid being adapted to releasably store an electrode tray on the underside of the lid.

In another aspect of the invention, there is provided a package for storing electrodes prior to use with a defibrillator, the package comprising: a substantially rigid tray defining a recess for receiving the electrodes; and a peel-off sheet releasably secured to the tray so as to hermetically seal the electrodes within the recess.

In another aspect of the invention, there is provided an electrode for use in applying an electric current to a patient, the electrode comprising: a hydrogel pad having a first generally rectangular shape with rounded corners; and

a conductor mounted to the hydrogel pad, the conductor having a second generally rectangular shape with rounded corners, with the footprint of the conductor being less that the footprint of the hydrogel pad, the conductor being configured at a first edge thereof to be connected to the circuit for applying the electric current to the patient, whereby when the conductor is mounted to the hydrogel pad, the hydrogel pad will overlap the conductor on at least the three remaining edges.

In another aspect of the invention, there is provided a defibrillator for applying a therapeutic shock pulse to a patient, the defibrillator having a footprint substantially the size of the footprint of its associated electrodes.

In another aspect of the invention, there is provided a defibrillator for applying a therapeutic shock pulse to a patient, the defibrillator comprising: a body enclosing hardware for generating the shock pulse, the body including a communication device for accessing the hardware without opening the body.

In another aspect of the invention, there is provided a defibrillator comprising a body that encloses hardware associated with the defibrillator, and further comprising a fault analysis system comprising a visual signal indicating whether or not there is a malfunction in the hardware, the visual signal being visible without opening the body.

In another aspect of the invention, there is provided an electrode tray, where electrodes in the tray are face-to-face on a release liner.

In another aspect of the invention, there is provided an electrode connector that includes a component that allows a defibrillator to detect when the connector is inserted.

In another aspect of the invention, there are provided electrodes in a package with an anode-to-cathode resistor, which: (a) allow device impedance circuit testing in periodic self-tests; (b) identify a unique electrode for shelf life duration monitoring; and (c) the resistor can be removed or modified by the periodic self-tests by using the defibrillator pulse when shelf life has expired.

In another aspect of the invention, there is provided the defibrillator waveform: (a) Pre-pulse detects: have thoracic impedance, detects impedance to real defibrillation currents, determines selection of capacitors, and determines waveform duration; (b) Pre-pulse is used to determine the control of a waveform greater that 250 joules;(c) Capacitor selection allows delivery of a pulse into thoracic impedance from 25-200 ohms without a change in capacitor voltage; (d) Capacitor selection allows delivery of a pulse into thoracic impedance from 25-200 ohms without inserting series resistors.

In another aspect of the invention, there is provided Defibrillation waveform electronics: (a) Patient-connected leads have leakage protection with semiconductors, from the defibrillator capacitor high voltage; (b) Patient-connected leads are protected from a second external defibrillator (damped sine wave or multi-phasic) with semiconductors; (c) Patient-connected leads are protected from ESD discharge with semiconductors; (d) Double fault protection of the defibrillator high voltage to the patient leads is provided with semiconductors; and (e) ECG monitoring allows +/−5 volt offset voltages while connected to the defibrillation high voltage capacitors.

In another aspect of the invention, there is provided defibrillator capacitor voltage is dumped with internal discharge electronics that utilize low power, low cost semiconductors.

In another aspect of the invention, there is provided Battery for device operation is sized to perform a single patient rescue sequence and be replaced for the next patient rescue.

In another aspect of the invention, there is provided Provide fault analysis, with transmission of results on external data communications output for remote monitoring device status.

In another aspect of the invention, there is provided capacitor charging with low voltage from battery combined with a safety dump circuit that stops dumping at a voltage just above the battery voltage.

In another aspect of the invention, there is provided a connector for detecting the nature of an associated electrode and its current use.

In another aspect of the invention, there is provided a battery for use in a defibrillator, the battery being sized for a single rescue event.

In another aspect of the invention, there is provided an automated external defibrillator comprising a key receptacle, with the defibrillator being configured for manual operation when the key receptacle is filled.

In another aspect of the invention, there is provided a defibrillator comprising a safety circuit having a shock delivery switch providing redundant control to the therapy delivery circuits.

In another aspect of the invention, there is provided a defibrillator wherein ECG monitoring and impedance monitoring utilize the same circuitry.

In another aspect of the invention, there is provided a defibrillator having an independent time base, and alarm activation, for initiating periodic self-testing.

In another aspect of the invention, there is provided a defibrillator adapted to provide continuous ECG analysis for detection of ventricular fibrillation during periods of operator contact with the patient for the purpose of expediting delivery of defibrillation shocks.

In another aspect of the invention, there is provided A defibrillator adapted to provide real-time coaching to a user during a rescue.

### BRIEF DESCRIPTION OF THE DRAWINGS

These and other objects and features of the present inventions will be more fully disclosed or rendered obvious by the following detailed description of the preferred embodiment of the invention, which is to be considered together with the accompanying drawings wherein like numbers refer to like parts and further wherein:

FIG. 1 is a schematic diagram illustrating how the novel automated external defibrillator (AED) system of the present invention addresses a significant market need;

FIG. 2 is a schematic diagram illustrating the novel AED system of the present invention applied to a patient;

FIG. 3 is a schematic diagram showing the distribution curve for thoracic impedance for a typical patient population;

FIG. 4 is a schematic diagram of a bi-phasic waveform useful in defibrillating the heart;

FIG. 5 is a schematic diagram of a bi-phasic waveform illustrating that the slope (or “tilt”) of the waveform is a function of patient impedance and defibrillator capacitance;

FIG. 6 is a schematic diagram showing how the slope (or “tilt”) of a bi-phasic waveform flattens with increasing patient impedance;

FIG. 7 is a schematic diagram showing how the slope (or “tilt”) of a bi-phasic waveform flattens with increasing defibrillator capacitance;

FIG. 8 is a schematic diagram showing the strength duration curve which illustrates the relationship between current, time and successful defibrillation;

FIG. 9 is a schematic diagram showing how the calculated average current level can be determined where the defibrillator has a sloping current curve (e.g., with a capacitance-generated pulse);

FIG. 10 is a schematic diagram illustrating how it is possible to widen the width of the shocking pulse, and thereby change the calculated average current level, by allowing the capacitance to discharge longer;

FIG. 11 is a schematic diagram illustrating how the gap between a 100 μF capacitance curve and the 50% successful defibrillation curve remains fairly constant regardless of how much the shock pulse is elongated;

FIG. 12 is a schematic diagram illustrating how the gap between a 200 μF capacitance curve and the 50% successful defibrillation curve increases as the shock pulse is elongated;

FIG. 13 is a schematic diagram illustrating one preferred technique for providing the bi-phasic waveform of the present invention;

FIG. 14 is a schematic illustration of the bi-phasic waveform produced by the present invention;

FIG. 15 is a perspective view of the novel AED system with its cover closed;

FIG. 16 is a perspective view of the novel AED system with its cover opened;

FIG. 17 is a view showing the front face of the system's defibrillator;

FIG. 18 is a view showing the defibrillator's battery slot and flashcard slot;

FIG. 19 is a view showing the defibrillator's battery;

FIG. 20 is a view showing the defibrillator's speaker, microphone, alarm and selected internal electronics;

FIG. 21 is a view showing the rear side of the defibrillator's front casing, showing the speaker ports, microphone ports and alarm ports;

FIG. 22 is a schematic diagram of the defibrillator system;

FIG. 23 is another schematic diagram of the defibrillator system;

FIG. 24 is a schematic diagram of the defibrillator's H-Bridge circuit;

FIG. 25 is a schematic diagram of the defibrillator's internal energy dump circuit;

FIG. 26 is a view showing the system's electrode package, with the package's sheet of sealing material having been removed from the tray;

FIG. 27 is a perspective view of one end of electrode package's tray, with the tray being shown at an intermediate stage of manufacture;

FIG. 28 is a perspective view showing the same end of the tray, with the tray being shown at a subsequent stage of manufacture;

FIG. 29 is a perspective view showing the other end of the tray;

FIG. 30 is a perspective view showing one end of the underside of the system's cover;

FIG. 31 is a perspective view showing the other end of the underside of the system's cover;

FIG. 32 is a view showing various elements used to construct the system's electrodes;

FIGS. 33-38 show various steps in the construction of the system's electrodes; and

FIG. 39 is a perspective view showing one preferred way to connect the electrodes to the tray.

### DETAILED DESCRIPTION OF THE PREFERRED EMBODIMENT

### Introduction

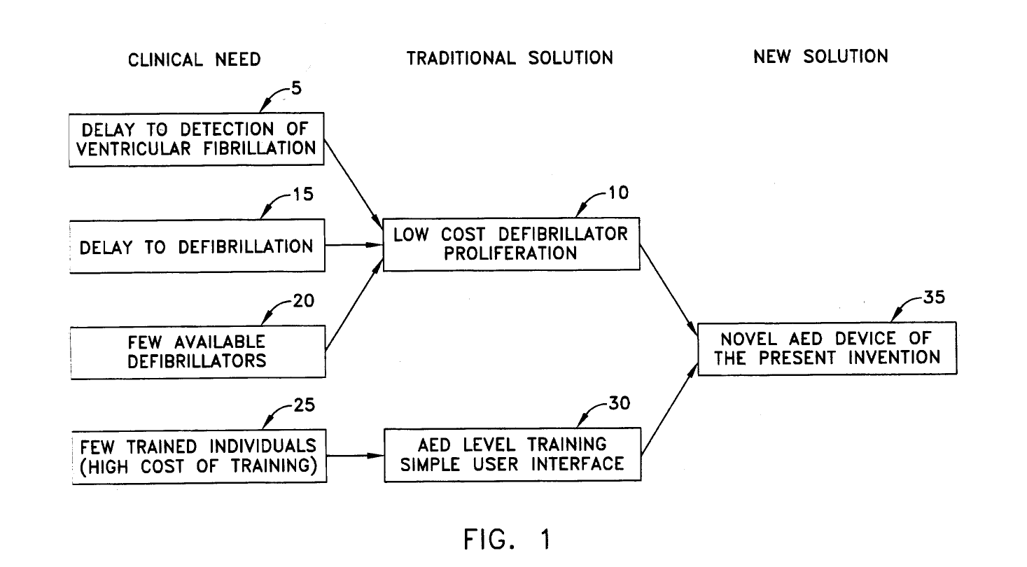

FIG. 1 illustrates various clinical needs associated with cardiac fibrillation and the proposed solutions for those clinical needs. Delays in the detection of ventricular fibrillation, 5, may be averted by a broad deployment of low cost defibrillators, 10. Similarly, a delay in defibrillation therapy, 15, and the availability of a limited number of defibrillators, 20, can be avoided by providing for a broad deployment of low cost defibrillators, 10. The problems associated with the high cost of training to use a defibrillator, 25, thereby resulting in fewer trained personnel, is overcome by automated external defibrillators (AED's).

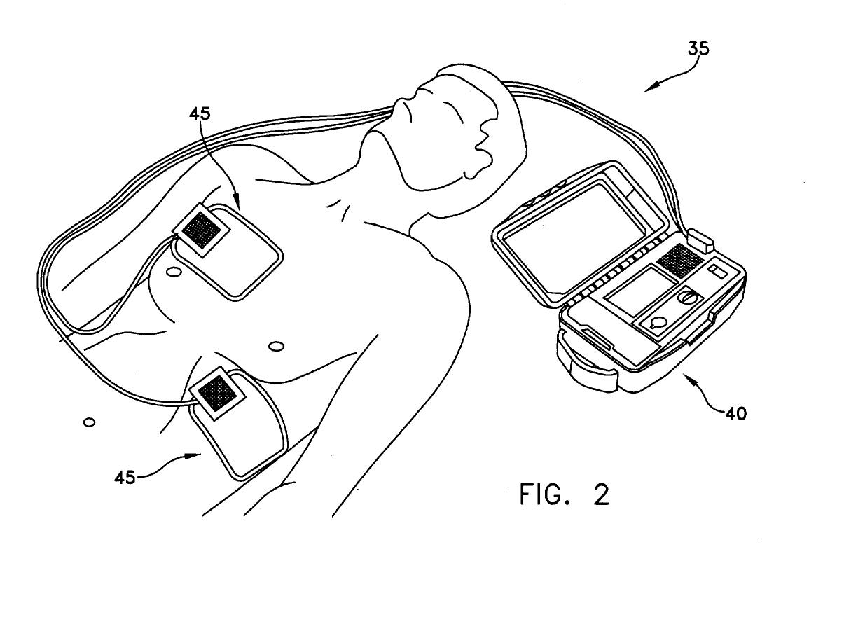

All these needs and solutions, as well as others, are addressed by the novel defibrillator system 35 (FIGS. 1 and 2) of the present invention, which comprises an automated external defibrillator (AED) 40 and a pair of associated electrodes 45.

### Defibrillator System 35 in General

As noted above, a defibrillator is designed to deliver a therapeutic electric shock to the heart in order to stop chaotic fibrillation and restore the normal heart rhythm. In order for successful defibrillation to be achieved, an external defibrillator must deliver sufficient voltage to overcome the thoracic impedance of the patient and sufficient current to provide the therapeutic effect to the heart muscle.

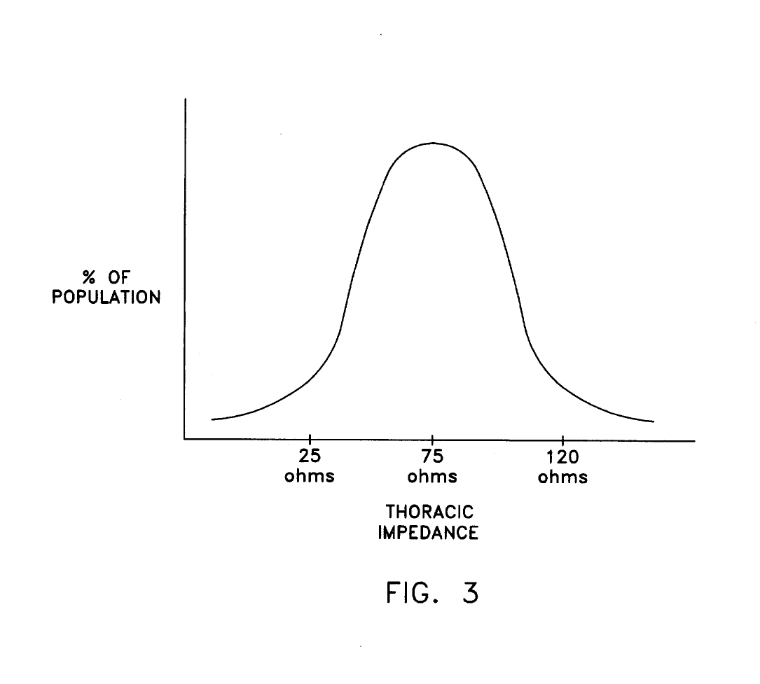

As also noted above, the level of thoracic impedance tends to vary from person to person. In a typical population, the distribution of thoracic impedance generally follows a bell-shaped curve. More particularly, and looking now at FIG. 3, the distribution curve for thoracic impedance is typically centered at about 75 ohms, with about 90% of the population falling in the range of between about 25 ohms and about 120 ohms.

In accordance with Ohm's law, if a defibrillating pulse of fixed voltage is applied to a patient, the level of current entering the patient will vary in accordance with the thoracic impedance of the patient. Unfortunately, this can present serious problems for patients who are at the low end of the impedance curve and for patients who are at the high end of the impedance curve. More particularly, for low impedance patients, the current delivered to the patient may go too high, which can result in burning or other tissue damage, including cardiac stunning and reduced efficacy; alternatively, for high impedance patients, the current delivered to the patient may go too low and fail to provide the desired therapeutic benefit, i.e., the heart will not be successfully defibrillated.

Therefore, it can be desirable to vary the voltage of the defibrillating pulse according to the impedance of the patient.

In general there are two steps associated with varying the defibrillating pulse according to the impedance of the patient: first, the impedance of the patient must be measured, and second, the voltage of the defibrillating pulse must be set according to the measured impedance of the patient.

In order for an external defibrillator to be used in the widest possible range of situations, the defibrillator should be portable. As a result, portable external defibrillators traditionally rely on batteries as their source of electrical energy. Since batteries are generally able to deliver only a limited voltage, most portable defibrillators use capacitors to accumulate charge from the battery and release it in the shocking pulse.

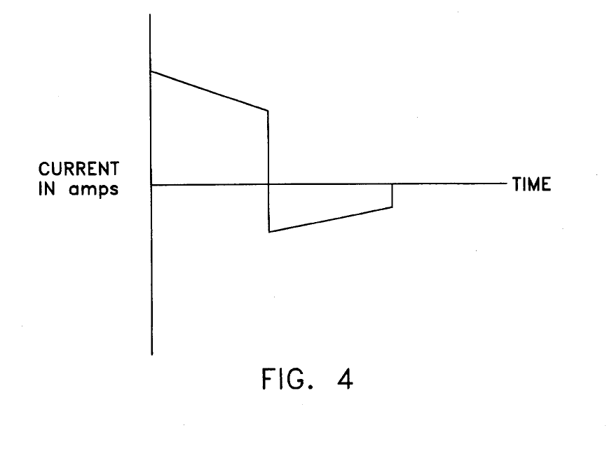

In addition to the foregoing, it has also been found that it can be therapeutically beneficial for the defibrillator to provide a multi-phase current. More particularly, and looking now at FIG. 4, it has been found that it can be therapeutically beneficial to provide a bi-phasic current profile for the shocking pulse.

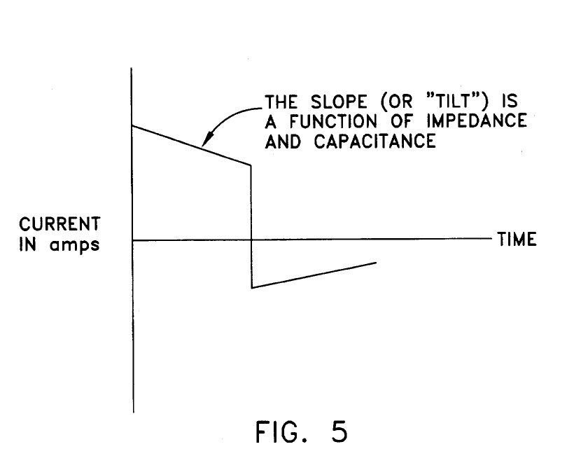

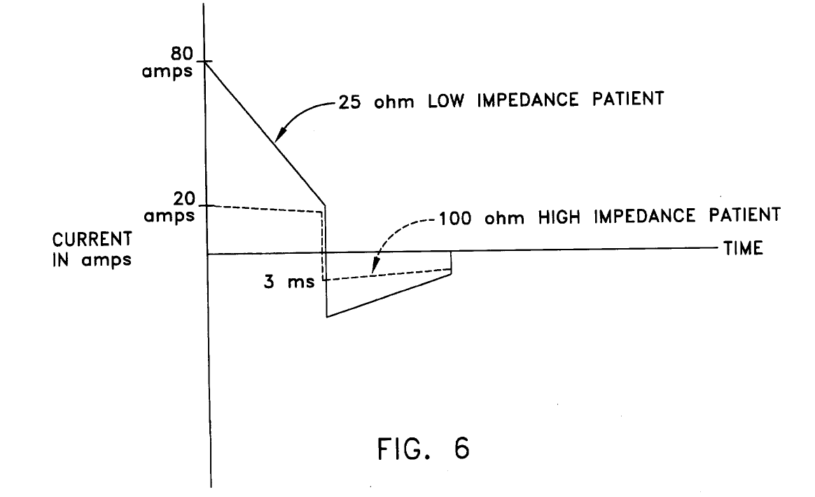

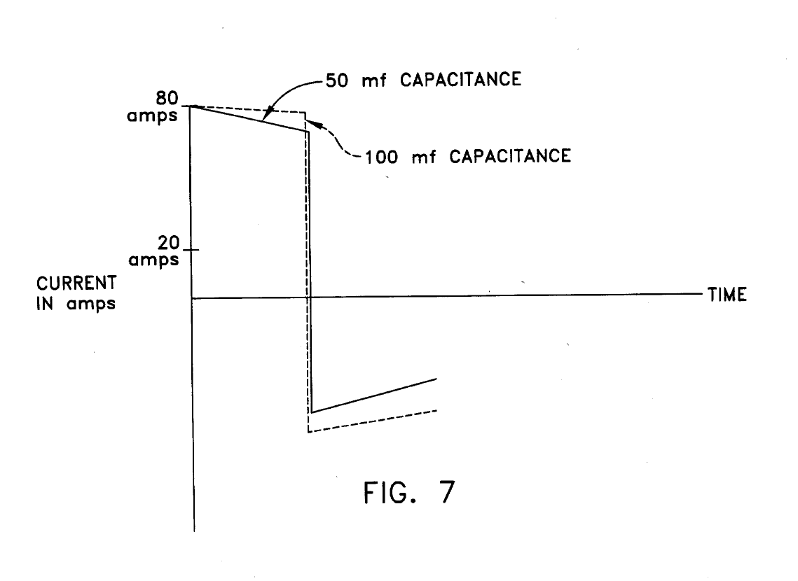

To the extent that the defibrillator uses capacitors to generate a bi-phasic waveform, the waveforms tend to have a slope (or “tilt”) that is a function of (i) the patient's thoracic impedance, and (2) the defibrillator's capacitance (FIG. 5). More particularly, and looking now at FIG. 6, for a fixed voltage defibrillator, an increase in thoracic impedance tends to reduce the tilt of the bi-phasic waveform. Similarly, and looking now at FIG. 7, for a fixed voltage defibrillator, an increase in capacitance tends to reduce the tilt of the bi-phasic waveform. Manufacturers have traditionally provided lower capacitance (e.g., 100 μF) in portable bi-phasic defibrillators for a variety of reasons: lower capacitance is generally lighter; lower capacitance is typically cheaper; and lower capacitance requires less energy from the battery, thus allowing smaller and lighter batteries to be used in the defibrillator.

Thus it will be appreciated that a given fixed voltage defibrillator will provide a bi-phasic waveform having different tilt profiles depending on the impedance of the patient and on the capacitance of the defibrillator.

It should also be appreciated that it is generally easier to defibrillate the low impedance patient, and harder to defibrillate the high impedance patient, since defibrillation requires a certain level of current and it can be difficult to provide that level of current in high impedance patients.

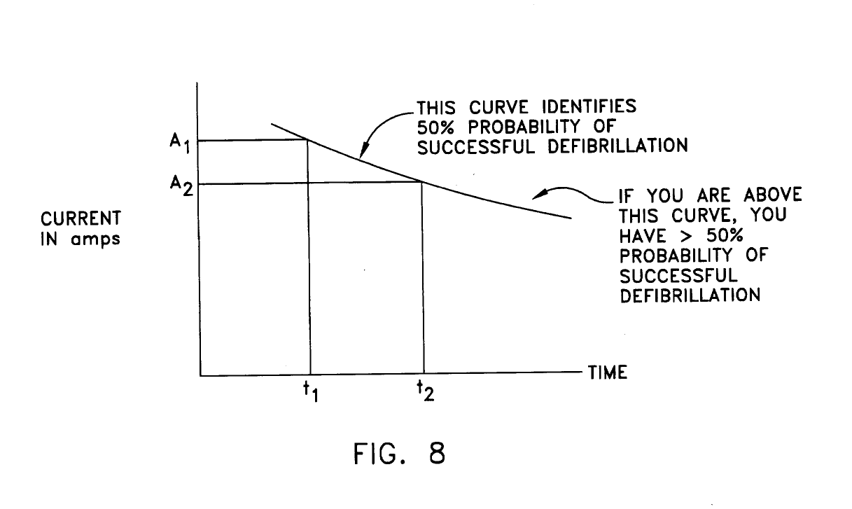

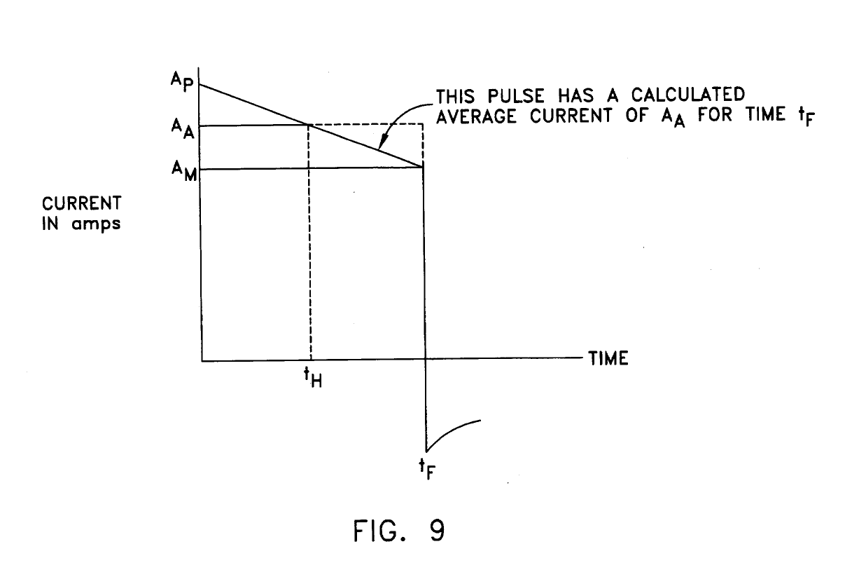

More particularly, research has shown that there is a strength duration curve which illustrates the relationship between current, time and successful defibrillation. Looking now at FIG. 8, it has been found that, for a given level of current, longer shock pulses increase the probability of successful defibrillation. Stated another way, in order to achieve a 50% probability of successful defibrillation, the defibrillator can either apply a current of level A₁ for a time t₁, or it can apply a current of level A₂ for a time t₁, where A₁>A₂ and t₂>t₁.

Where the defibrillator has a sloping current curve (e.g., with a capacitance-generated pulse), the effective current level can be considered to be the calculated average current level. More particularly, with the capacitance-generated pulse shown in FIG. 9, the pulse is considered to have a calculated average current of level A_{A} for time t_{F}.

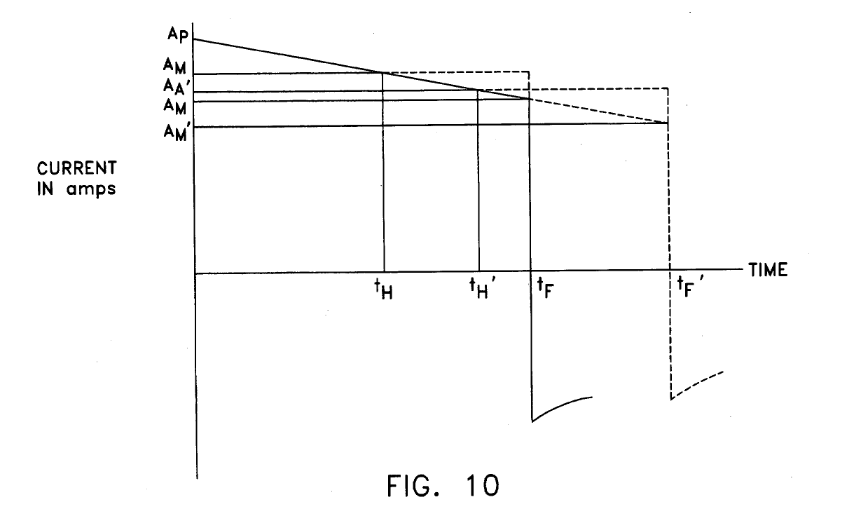

Thus, when a defibrillator uses a 100 μF capacitance to generate its shocking pulse, the defibrillator will have an effective current curve which looks something like that shown in FIG. 10, depending on how long the capacitance is allowed to discharge. For example, if the 100 μF capacitance is discharged for time t_{F}, it will yield an effective current of A_{A} amps; or if the same capacitance is discharged for a longer period t_{F}′, it will yield an effective current of A_{A}′ amps.

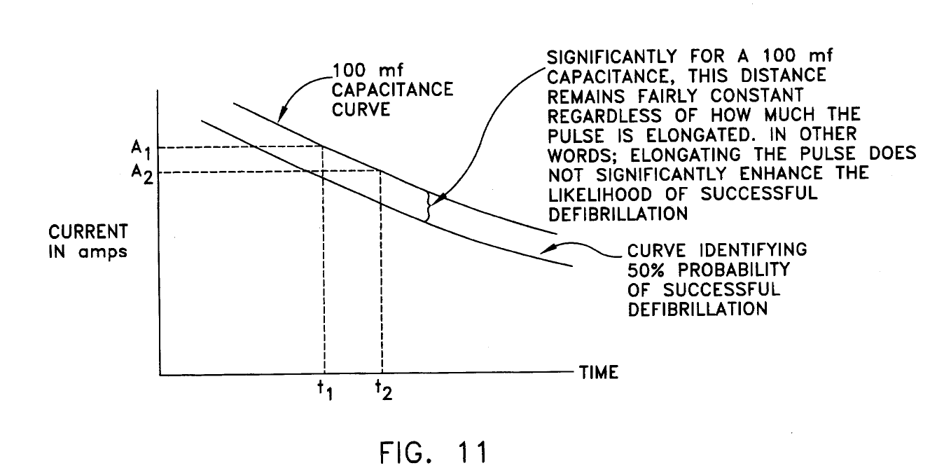

Significantly, it has now been discovered that when the calculated average current level of a 100 μF capacitance is plotted against successful defibrillation, that there is a fairly constant relationship. More particularly, and looking now at FIG. 11, it has been found that for a 100 μF capacitance, the gap between the 100 μF capacitance curve and the 50% successful defibrillation curve remains fairly constant regardless of how much the pulse is elongated. In other words, elongating the pulse does not significantly enhance the likelihood of successful defibrillation when generating the shocking pulse using 100 μF capacitance.

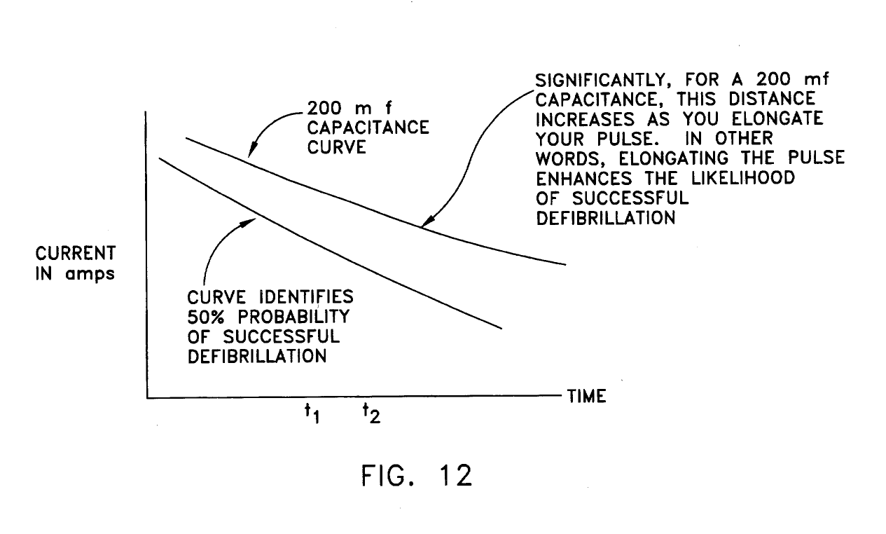

However, a larger capacitance has a different pulse profile than a 100 μF capacitance. Significantly, it has now been discovered that when the calculated average current level of the 200 μF capacitance is plotted against successful defibrillation, there is a diverging relationship. More particularly, and looking now at FIG. 12, for a 200 μF capacitance, the gap between the 200 μF capacitance curve and the 50% successful defibrillation curve increases as the pulse is elongated. In other words, elongating the pulse enhances the likelihood of successful defibrillation when generating the shocking pulse using 200 μF capacitance.

Therefore, even though manufacturers have traditionally provided lower (e.g., 100 μF) capacitance in portable bi-phasic defibrillators, it has now been discovered that there is a significant advantage to providing higher (e.g., 200 μF) capacitance in a portable bi-phasic defibrillator. Thus, in the preferred embodiment of the present invention, defibrillator system 35 comprises a portable bi-phasic defibrillator having a higher (e.g., 200 μF) capacitance.

In essence, with the present invention, it has been discovered that a significantly more effective bi-phasic defibrillator can be constructed by configuring the defibrillator so that for higher impedance patients, it (1) has a higher capacitance (e.g., 200 μF) so that it has a reduced tilt to its waveform, whereby to obtain a higher calculated average current level for a similar shock voltage, and (2) has an elongated pulse width, whereby providing a higher defibrillation efficiency.

On the other hand, for low impedance patients, the defibrillator is configured so as to limit the current and thereby reduce cardiac stunning and post shock arrhythmias. This is preferably done by controlling the shape of the defibrillation pulse, by applying a lower voltage with a larger effective capacitance.

Furthermore, it has been discovered that a bi-phasic waveform having a phase 2:phase 1 charge ratio of approximately 0.38 is most efficacious. In this respect it should be appreciated that in the context of FIG. 4, the charge ratio can be thought of as the ratio of the bordered area of phase 2 divided by the bordered area of phase 1.

Preferably the applied voltage and the higher (e.g., 200 μF) effective capacitance is provided by a bank of individual capacitors, at least some of which are stacked 2 high, all charged to a common voltage and switchable so as to configure a desired discharge.

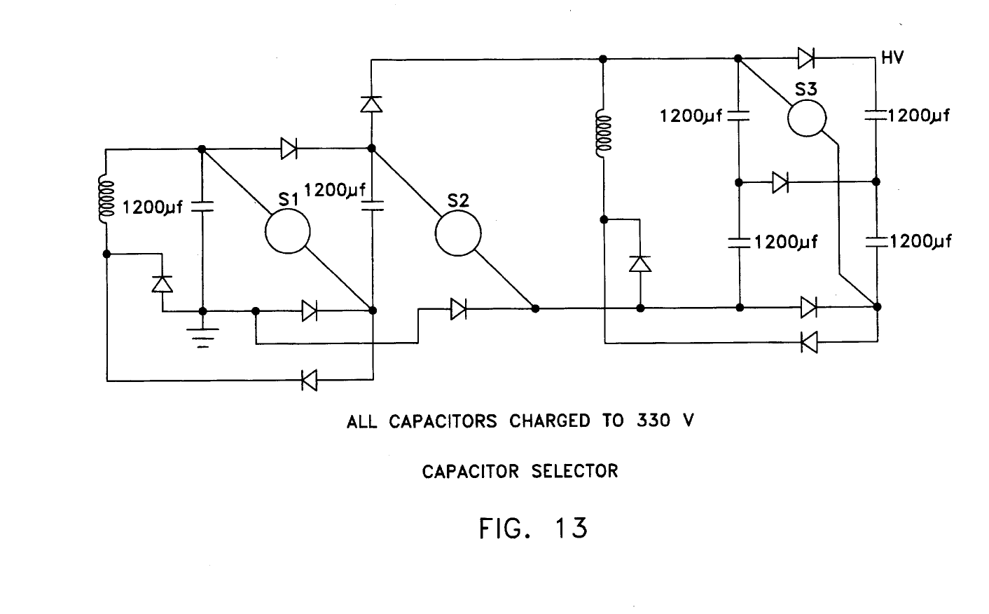

In one preferred form of the invention, and looking now at FIG. 13, there are 6 capacitors, arranged in a 1-1-2-2 configuration and charged to 330 volts each. Depending on the state of switches S1, S2 and S3, anywhere from 2-6 capacitors may be fired, so as to provide a voltage of 660-1980 volts.

More particularly, the capacitor circuit shown in FIG. 13 comprises six 1200 μF capacitors which, depending on the state of switches S1, S2 and S3, can be configured to provide a range of voltages with differing capacitances. The following table shows some of the possible configurations for the capacitor circuit:

| | | | Number of | | |
|---|---|---|---|---|---|
| Switch | Switch | Switch | Capacitors | Shock | Effective |
| S1 | S2 | S3 | Fired | Voltage | Capacitance |
| On | Off | Off | 2 | 660 V | 1800 μF |
| Off | On | Off | 3 | 990 V | 800 μF |
| Off | On | Off | 4 | 1320 V | 400 μF |
| Off | On | On | 5 | 1650 V | 266 μF |
| On | On | On | 6 | 1980 V | 200 μF |

Thus it will be seen that the capacitor circuit shown in FIG. 13 comprises 6 1200 μF capacitors, all charged to a fixed charge voltage of 330 volts, and depending on the state of its three switches S1, S2 and S3, is capable of providing anywhere from 660 to 1980 volts, at a capacitance of anywhere from 1800 μF to 200 μF. Significantly, a minimum of 200 μF is maintained even when all 6 capacitors are fired.

In order to provide an appropriate therapeutic shock to the patient, it is necessary to know the thoracic impedance of the patient. The present invention provides a unique approach for doing this. More particularly, in accordance with another aspect of the present invention, the defibrillator is configured to initially discharge, very briefly, 1 set of 2 stacked capacitors so as to generate a “pre-pulse”. By using just 2 of the 6 stacked capacitors, this pre-pulse has a voltage which is low enough (e.g., 660 volts) to avoid harming a patient having a low thoracic impedance (e.g., 25 ohms), since current flow will generally be under 30 amps (e.g., 660 volts/25 ohms=26 amps). This pre-pulse has a duration long enough to obtain an accurate reading of the patient's thoracic impedance due to electrode-to-skin interface effects, but short enough to avoid substantially depleting the capacitors. No therapeutic effect is rendered to the patient during the pre-pulse, and the pre-pulse is terminated prior to applying the subsequent therapeutic pulse (see below). In one preferred form of the invention, the pre-pulse is approximately 100 μseconds to 1 millisecond in duration to resolve the correct prediction of patient impedance.

Of course, it will be appreciated that the level of current of the pre-pulse will vary in accordance with the thoracic impedance of the patient. Thus, where the defibrillator is configured to generate a 660 volt pre-pulse, and the patient has a low thoracic impedance (e.g., 25 ohms), the current flow will generally be under 30 amps (e.g., 660 volts/25 ohms=26 amps); and where the patient has a high thoracic impedance (e.g., 120 ohms), the current flow will generally be under 3 amps (e.g., 600 volts/120 ohms=3 amps). Of course, the pre-pulse current level must be sufficient to predict patient impedance.

Once the pre-pulse has been used to identify the thoracic impedance of the patient, the unit is ready to apply the therapeutic shock to the patient. More particularly, once the defibrillator has identified the thoracic impedance of patient, it can determine how much voltage to apply to that patient in order to provide the appropriate therapeutic shock, the duration of the pulse and the desired shape of the pulse. The defibrillator then determines how many of the capacitors to fire in order to achieve the desired shock voltage (and hence the desired shock current), and then fires that number of capacitors for the desired pulse width. By choosing exactly how many capacitors are fired, the level of voltage applied to the patient can be regulated, and thus the level of current applied to the patient can be regulated. As a result, the defibrillator can avoid applying too much current to patients having a low thoracic impedance while still ensuring that an effective shocking pulse is delivered to the patient.

Thus, with the preferred embodiment of the present invention, which is intended to use the capacitor construction of FIG. 13, a microprocessor uses a lookup table to determine how many microprocessors to fire when providing the shocking pulse. This lookup table is preferably as follows:

| Measured | | Number of | | | |
|---|---|---|---|---|---|
| Patient | | Capacitors | Shock | Peak | |
| Impedance | | To Fire | Voltage | Current | Capacitance |
| 25-32 | ohms | 2 | 660 V | 26-21 A | 1800 μF |
| 33-44 | ohms | 3 | 990 V | 30-23 A | 800 μF |
| 45-54 | ohms | 4 | 1320 V | 29-24 A | 400 μF |
| 55-62 | ohms | 5* | 1650 V | 30-27 A | 266 μF |
| 63-200 | ohms | 6** | 1980 V | 31-10 A | 200 μF |

*4 at 200 J, 5 at 360 J;
**5 at 63-100 ohms at 200 J, 6 at 101-200 ohms for 200 J; 6 at 63-100 at 360 J

thus, for higher impedance patients, higher average current is provided for greater efficacy.

Thus it will be seen that the defibrillator can vary the voltage of the shocking pulse according to the measured impedance of the patient, so as to ensure that an adequate voltage and amperage is applied to the patient, without applying too much current to the patient; and the defibrillator always provides at least 200 μF of capacitance, so as to ensure that the advantages of a 200 μF pulse profile is obtained.

Significantly, the present invention also provides a capacitance which varies in inverse proportion to the measured patient impedance, i.e., the defibrillator provides high capacitance for low patient impedance, and less capacitance for high patient impedance.

In an alternative form of the invention, the voltage and effective capacitance being applied to the patient can be regulated by selectively inserting (e.g., by appropriate switching) resistors into the waveform circuit.

Thus, with the present invention, the defibrillator is configured to (1) take energy out of one or more batteries; (2) store that energy into some number of capacitors; (3) pre-pulse the patient, using at least one of the capacitors, so as to test the thoracic impedance of the patient; (4) after determining the specific impedance of the patient, calculate the voltage to be applied to the patient; and (5) fire the appropriate number of capacitors to provide the desired shock pulse. Significantly, in the preferred form of the invention, the defibrillator's capacitance is provided by a fixed charge voltage, and the waveform's current is controlled by capacitance.

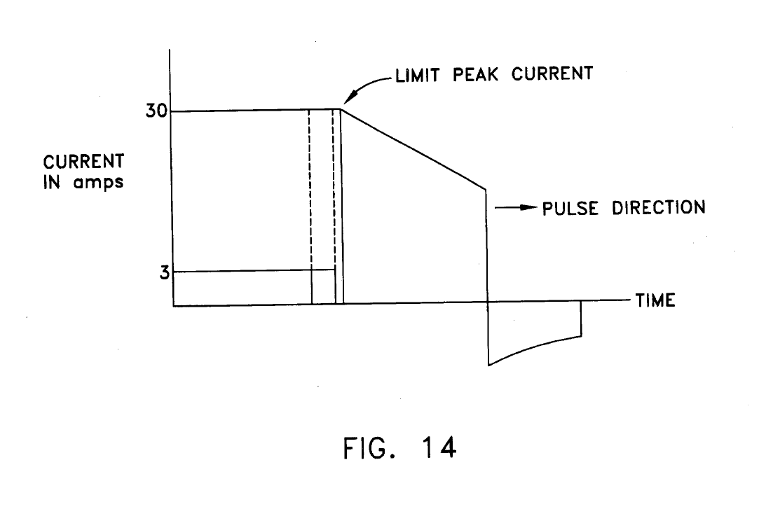

FIG. 14 is a schematic illustration of the bi-phasic waveform produced by defibrillator system 35.

### Defibrillator 40

Defibrillator 40 is compact, rugged, inexpensive, easy to use, water-resistant and electronically efficient. Defibrillator 40 is lightweight, weighing less than 6 pounds, and preferably weighing less than 3 pounds. Defibrillator 40 has an expected field life of 5 years.

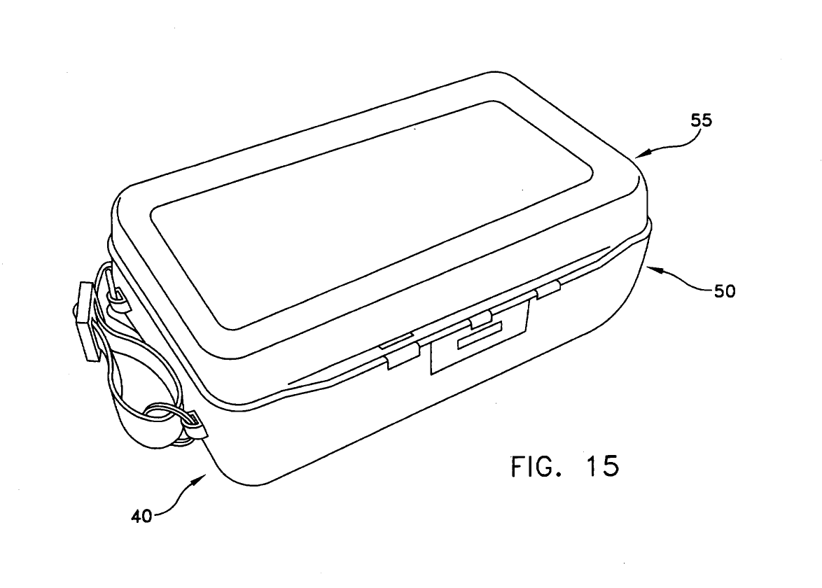

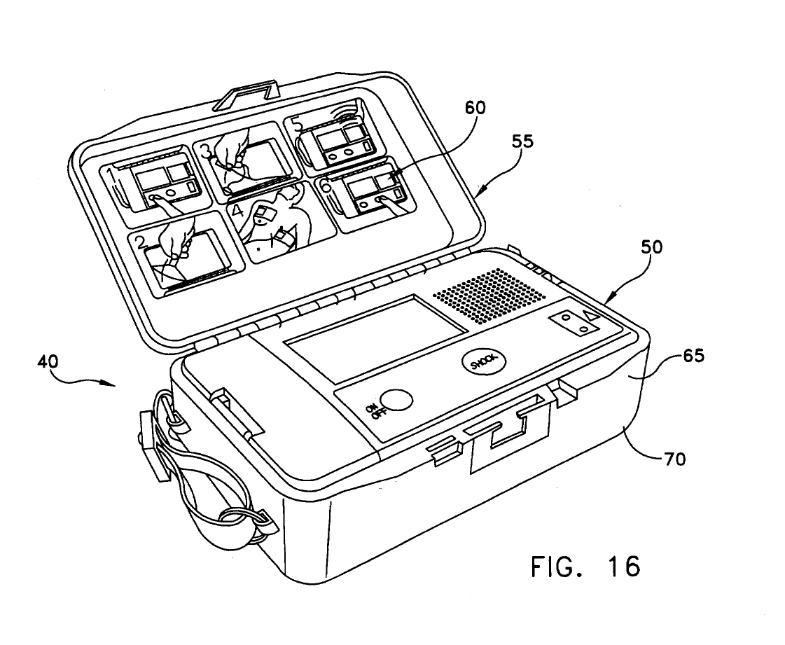

Looking now at FIGS. 15 and 16, in its preferred embodiment, defibrillator 40 is in the form of a unit (or box) comprising a body 50 and a lid 55. Body 50, which may be partially or completely coated with rubber and/or rubber-like materials, houses the electronics associated with the defibrillator. Lid 55 provides a cover for the top of body 50 and houses an electrode package 60 (FIG. 4) containing electrodes 45 (FIG. 2), as will hereinafter be discussed in further detail.

Body 50 houses the electronic hardware and software associated with the defibrillator. Body 50 comprises a front casing 65 (FIG. 16) and a back cover 70. In general, the configuration of body 50 is specifically designed to provide high voltage separation.

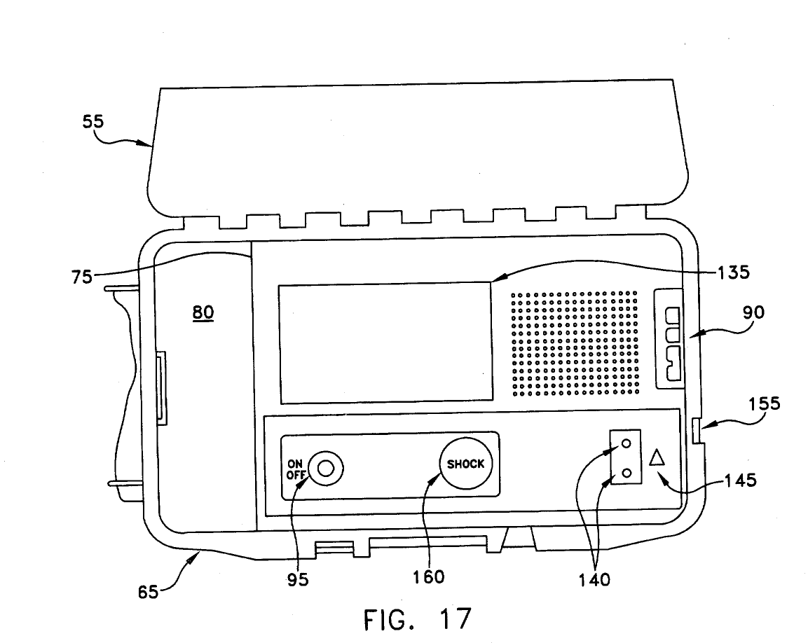

Looking next at FIGS. 17-21, front casing 65 comprises the following components:

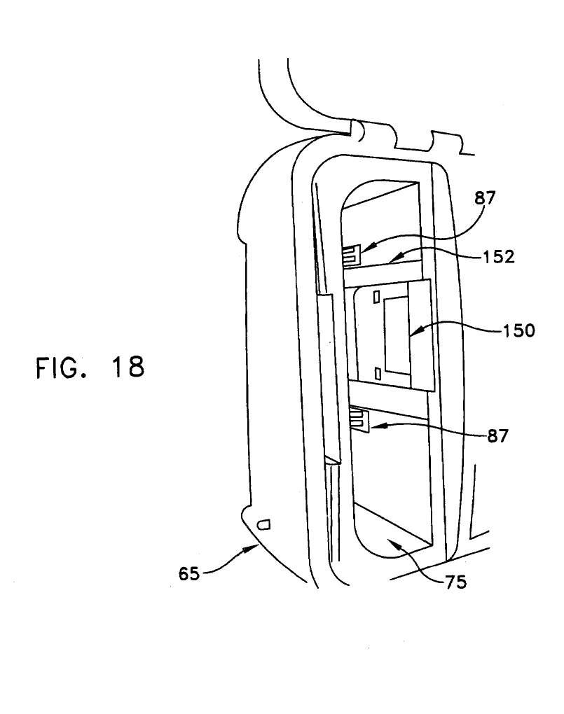

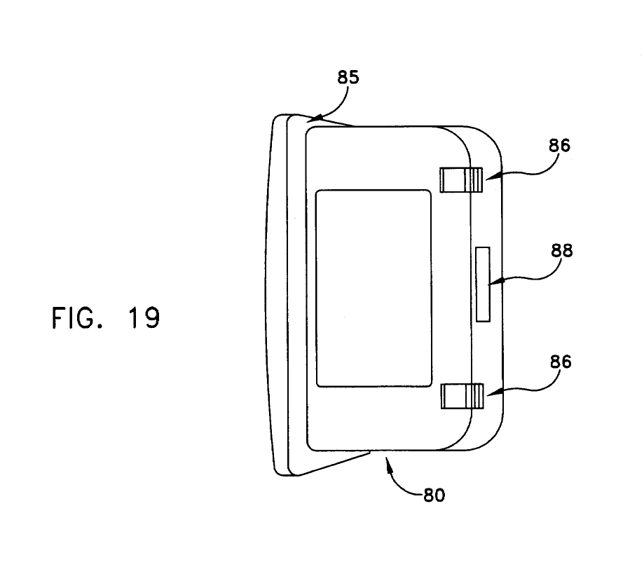

1. Battery Slot 75 and Battery 80: Body 50 includes a battery slot 75 (FIGS. 17 and 18) which receives a battery 80 (FIGS. 17 and 19), such as a Lithium Manganese Dioxide battery. In this respect it should be appreciated that as used herein, the term “battery” is intended to encompass a single cell construction or a multiple cell construction. Battery 80 includes a peripheral gasket 85 (FIG. 19) so that a substantially watertight seal will be formed when battery 80 is inserted in battery slot 75. Preferably the bottom of battery 80 makes a male-female engagement with the floor of battery slot 75 so as to ensure reliable engagement of battery contacts 86 (FIG. 19) with battery slot contacts 87 (FIG. 18). By way of example but not limitation, the bottom of battery 80 may include a recess 88 (FIG. 19) to receive a mating post (not shown) extending out of the floor of battery slot 75.

2. Connector Slot 90: The pair of electrodes 45 (FIG. 2) that are to be used in conjunction with defibrillator 40 are linked to body 50 via a connector (see below) that plugs into a connector slot 90 (FIG. 17). The electrodes are preferably connected to body 50 at the time of use. Alternatively, in order to save time during an emergency, the electrodes may be pre-plugged (or “pre-connected”) into connector slot 90.

3. Power Button 95: Unlike many prior art defibrillators that power up upon opening, defibrillator 40 is not automatically turned on when opened by the user. Instead, there is a power button 95 (FIG. 17) that needs to be pushed for the defibrillator unit to be activated.

4. Test Status Indicator 100: Defibrillator 40 is equipped with a fault analysis system that helps detect malfunctions associated with the unit. In the instance of a detected malfunction, the unit has a visual indicator or a test status indicator (e.g., an LED) 100 (FIG. 17) that can be observed by the user (e.g., an EMT, hospital nurse, etc.), thereby informing the user that the unit is malfunctional and needs to be replaced.

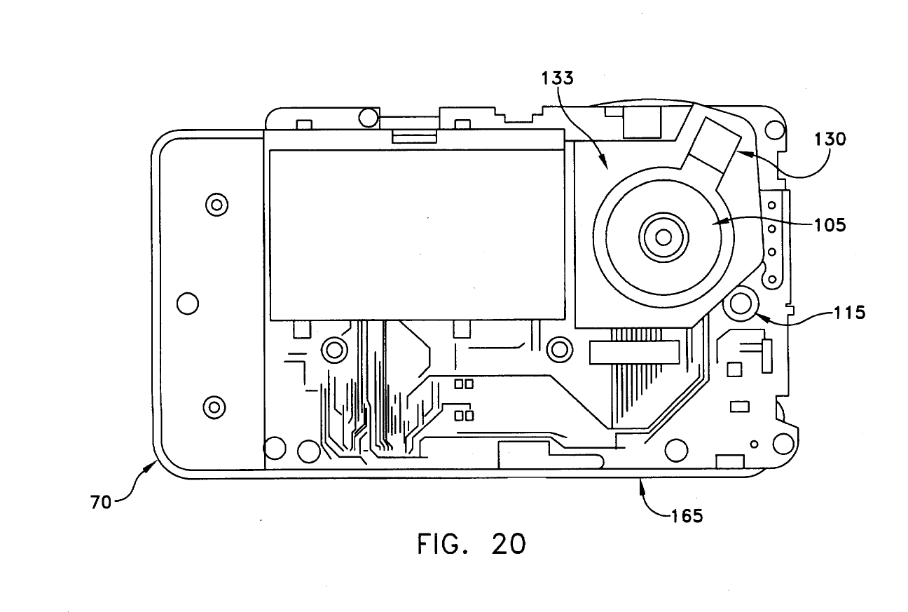

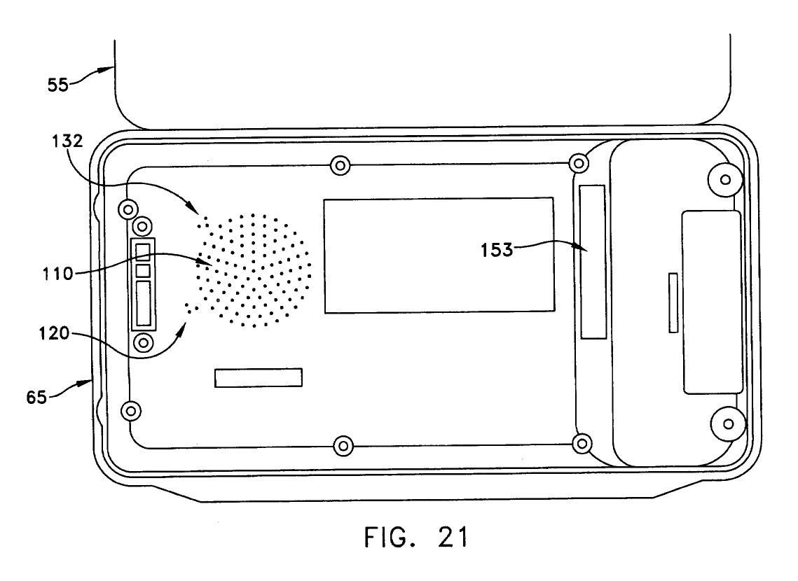

5. Voice System: When the defibrillator is turned on, a voice prompt that can be heard over speaker 105 (FIG. 20) accessed through speaker ports 110 (FIG. 21) guides the user through the American Heart Association® (AHA) ABCD sequence of evaluating the patient's condition. If it appears that defibrillation is required, the electrodes 45 are available for placement on the victim. The defibrillation method of the present invention involves a two-step process of applying the electrodes and pressing a single button (shock button 160, discussed below) for resuscitation. Speaker 105 also allows the user to be provided with instructions from a remote source (e.g., a hospital) over a radio link. A microphone 115 (FIG. 20) accessed through microphone ports 120 (FIG. 21) allows sounds at the emergency site to be recorded by the unit or transmitted to a remote site (e.g., a hospital) via a radio link or other types of communication links (e.g., cell phone, etc.). An alarm 130 (FIG. 20) accessed through alarm ports 132 (FIG. 21), allows an alarm to be sounded to the user. In order to render the defibrillator more water-resistant, it is preferred that a hydrophobic membrane (not shown) be placed between speaker 105 and speaker ports 110, between microphone 115 and microphone ports 120, and between alarm 130 and alarm ports 132. By way of example but not limitation, such a hydrophobic membrane may comprise Palilflex TL-1741 membrane material. In one preferred embodiment of the present invention, speaker 105, microphone 115 and/or alarm 130 are all mounted directly to a circuit board, one or more gaskets 133 (FIG. 20) are positioned about the elements, and then one or more hydrophobic membranes are placed between the gaskets and the ports, whereby to provide a substantially water-resistant unit.

6. LCD Display 135: The unit comprises a liquid crystal display (LCD) 135 (FIG. 17) that displays pertinent information such as electrocardiogram (ECG) graphs and also helps guide the user. The LCD display of the preferred embodiment is a 4-line display, but it is not intended to be limited thereto. Scroll buttons 140 (FIG. 17) are provided to facilitate presentation of text and/or graphics on LCD display 135. In the event that more information must be delivered to the user than can be conveniently displayed on LCD 135, a universal alert (comprising, for example, an LED) 145 (FIG. 17) can be activated (e.g., lit) so as to advise the user to consult the user manual which accompanies the system.

7. Flashcard Slot 150: In a preferred form of the invention, defibrillator 40 further comprises a data recording capability which records data (such as ECG data related to various cardiac parameters) related to every defibrillation event. Flashcard slot 150 (FIG. 18) is preferably used for transferring this stored data to a flash memory card. Preferably flashcard slot 150 is protected from electrostatic discharge (ESD) with an insulating membrane, e.g., a 0.005 polycarbonate sheet 152 (FIG. 18). The configuration of the battery and battery well also helps protect flashcard slot 150 from ESD. Preferably the PC board 153 (FIG. 21) containing the electrical contacts for battery 80 and flashcard slot 150 is sealed (e.g., with a gasket or other sealant) to front casing 65, whereby to permit electrical contact between the interior of battery slot 75 and the interior of front casing 65 without permitting water to pass therebetween. The contents of a flash memory card located in flashcard slot 150 may also be retrieved by the system to configure device operating parameters and manual defibrillation control button operation. If desired, an infrared (IR) port 155 (FIG. 17) may also be used to transfer information out of, or into, the defibrillator. And in one preferred form of the invention, IR port 155 can be used to collect fault analysis data from the defibrillator, e.g., a group of defibrillators 35 stored for use (e.g., in an ambulance or in an airliner) may be quickly and easily queried as to their fault status using their IR ports 155.

8. Shock Button 160: When electrodes 45 have been applied to the patient and the defibrillator's internal electronics determine that the patient is in need of cardiac defibrillation, the defibrillator prompts the user to depress shock button 160 (FIG. 17) so as to generate an electro-therapeutic pulse to stop fibrillation and restore the normal heart rhythm. Alternatively, in another embodiment, a medically trained individual could use shock button 160 to manually generate the defibrillating electric pulse.

9. Defibrillator Electronics 165: Defibrillator 40 comprises internal electronics 165 (FIG. 20) that preferably use the aforementioned stacked, switched capacitor design to generate an electric pulse having the desired bi-phasic waveform. The use of mechanical relays is preferably avoided. The defibrillator is preferably configured to deliver a maximum of 10 defibrillation shocks with a single primary cell battery. Defibrillator 40 is preferably a 200/360 J (joule) biphasic escalating energy defibrillator which provides the best clinically effective defibrillation therapy.

More particularly, the internal electronics 165 of defibrillator 40 are preferably adapted to generate a bi-phasic waveform of escalating energy, with 200 J being delivered on the first shock and 360 J being delivered on subsequent shocks. The defibrillator is specifically designed to limit peak current in low impedance patients so as to avoid injury.

As discussed above, defibrillator electronics 165, residing on one or more PC boards, preferably comprise a plurality of capacitors configured in a stacked, switchable configuration. The system is designed to (i) charge its capacitors to a fixed charge voltage, (ii) generate a very brief pre-pulse, using the energy stored in one pair of stacked capacitors, (iii) measure the impedance of the patient using the pre-pulse, and (iv) apply an appropriate bi-phasic waveform to the patient so as to defibrillate. By generating a very brief pre-pulse using the energy stored in one pair of stacked capacitors, the electric pulse will be brief enough, and generate a low enough amperage, to avoid harming the patient with excess current. At the same time, the application of the pre-pulse to the patient will allow the impedance of the patient to be measured using exactly the same type of current which will be applied to the patient during defibrillation, thus allowing for more accurate impedance measurements. Furthermore, by limiting the pre-pulse to a very brief duration, the stacked capacitors will be left substantially fully charged for the subsequent defibrillating shock; thus, the pre-pulse will enable a very accurate measurement of impedance without delaying application of the defibrillation shock.

Additionally, by using switches to select precisely which of the stacked capacitors are fired, an appropriate bi-phasic waveform can be generated for a particular patient.

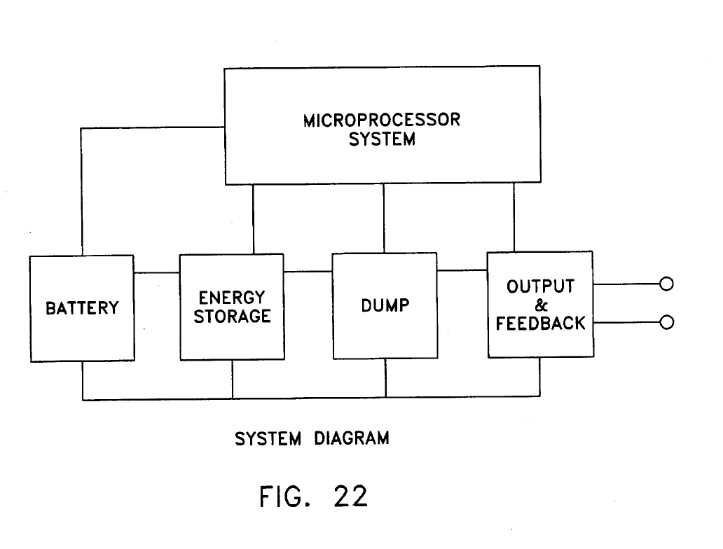

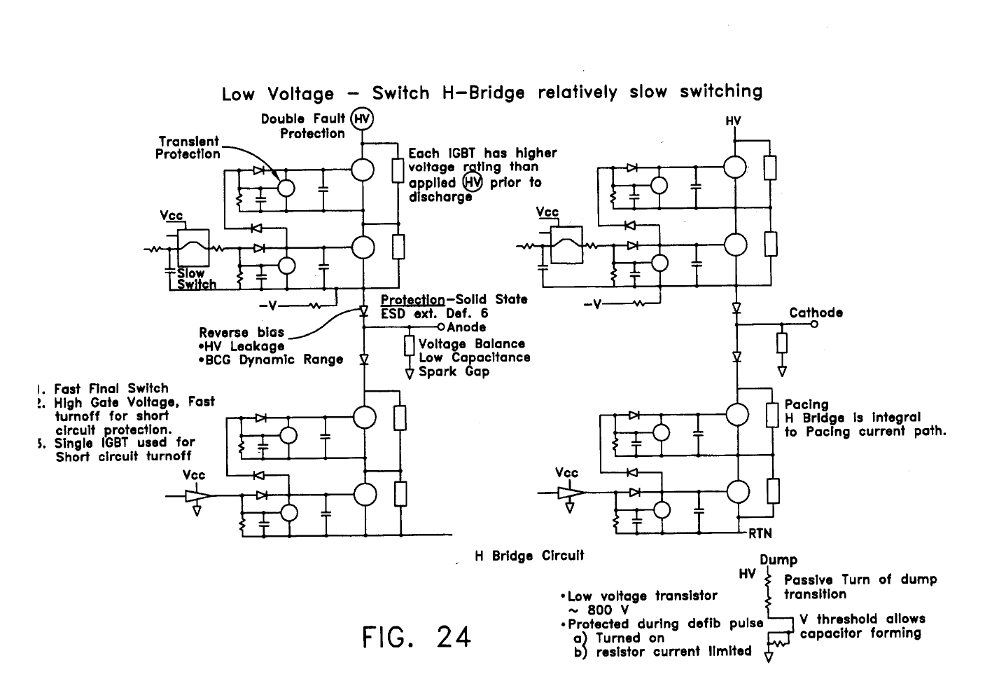

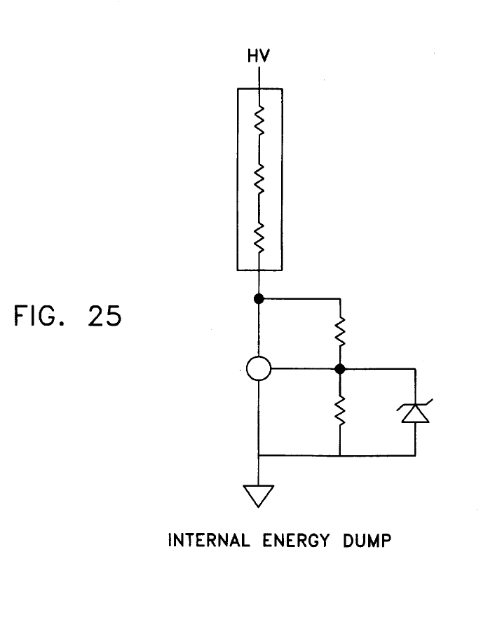

Further details regarding the internal electronics 165 of defibrillator 40, and the bi-phasic waveform generated thereby, are disclosed in FIGS. 22-25.

In the preferred embodiment, defibrillator 40 is insulated and is radiation shielded, including radio frequency (RF) shielded. And the defibrillator is preferably constructed so as to be resistant to a wide range of damped sine wave or bi-phasic external defibrillation shocks which may be applied to a patient while the defibrillator of the present invention is also connected to the same patient. In addition, the defibrillator is also constructed to be shock resistant, e.g., the circuit boards are rigidly mounted to front casing 65 so as to guard against mechanical shocks and vibrations.

### Electrodes 45

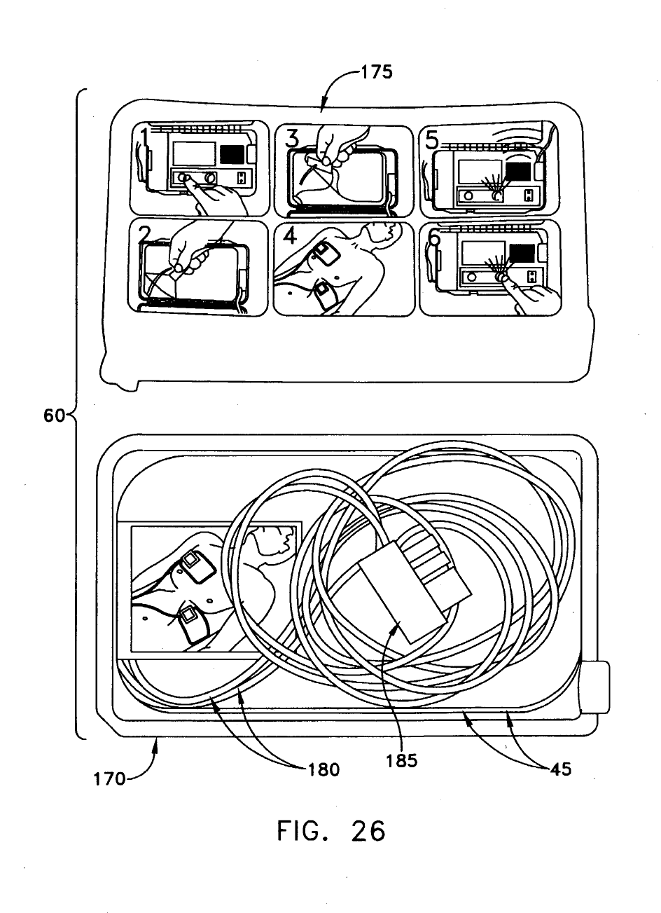

As discussed above, the defibrillator's lid 55 (FIG. 16) preferably stores the system's electrodes 45 (FIG. 2) prior to use. Furthermore, the electrodes are preferably housed in an electrode package 60 (FIG. 16) prior to use. More particularly, and looking now at FIG. 26, electrode package 60 preferably comprises a tray 170 and a peel-off top 175. Tray 170 and peel-off top 175 together form a substantially water-tight enclosure for housing the pair of electrodes 45, their respective leads 180 and a connector 185 which is used to connect electrode leads 180 to the defibrillator's connector slot 90 (FIG. 17). In one preferred form of the invention, connector 185 include resistors which can be used to uniquely identify the associated electrodes, e.g., a resistor of one value could identify an adult electrode, a resistor of another value could identify a pediatric electrode, etc.

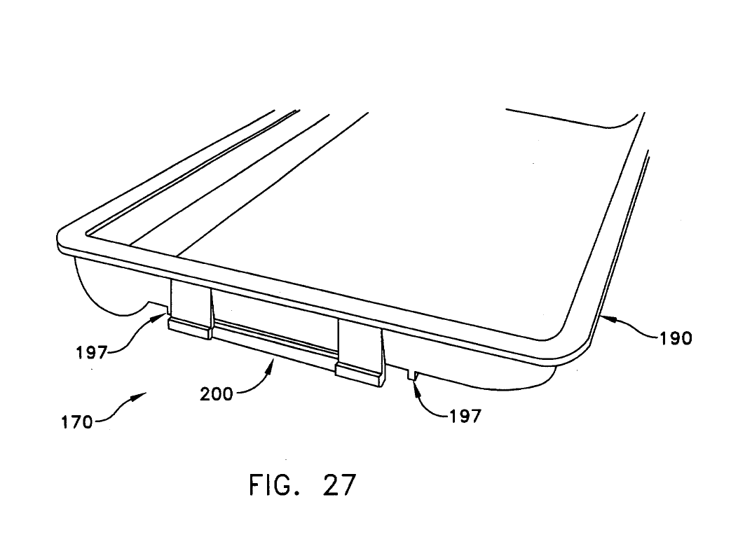

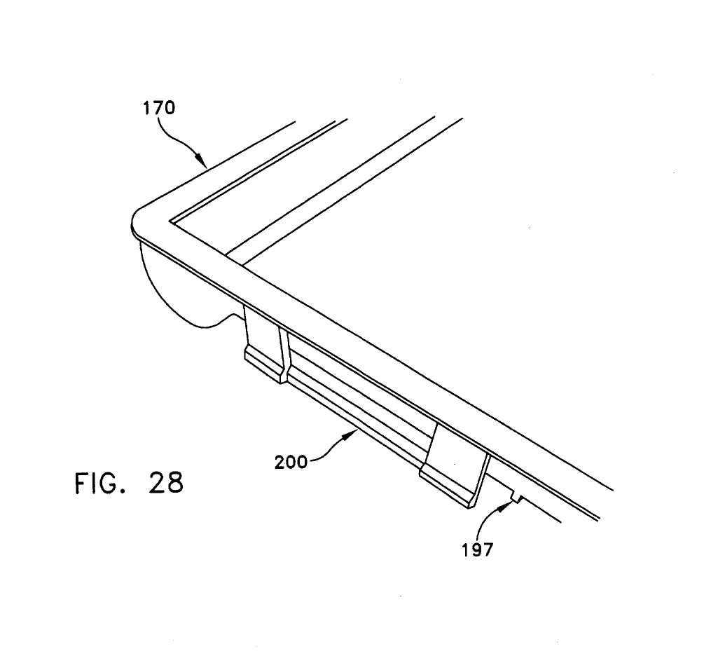

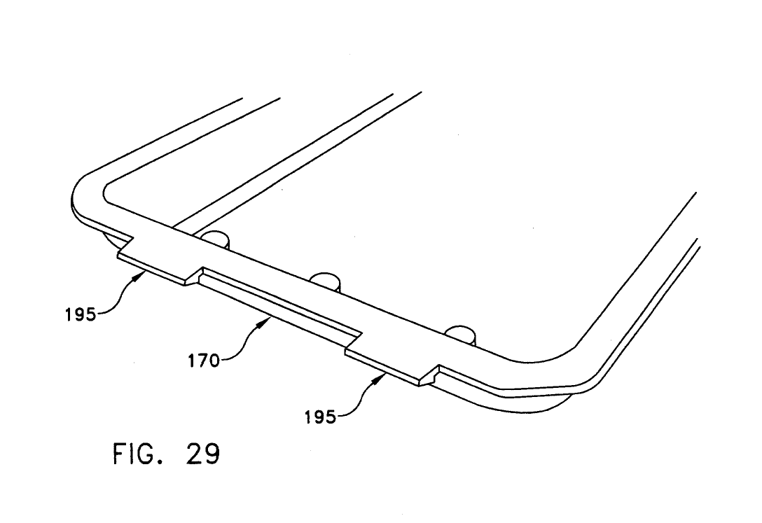

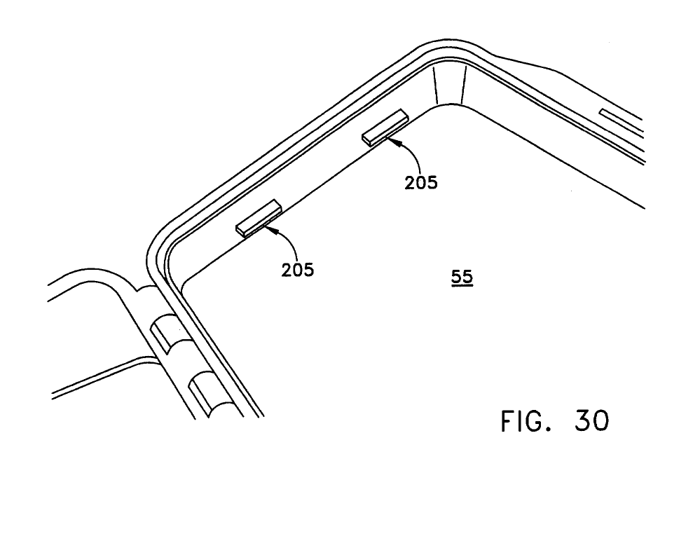

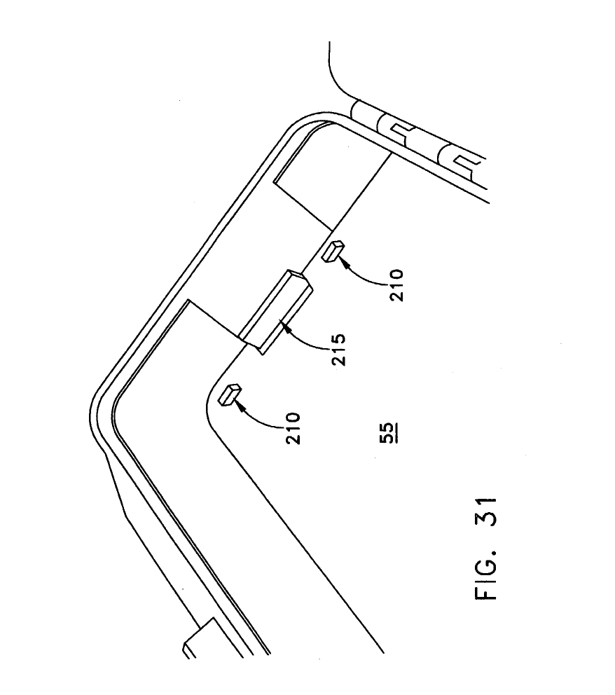

Preferably tray 170 is a semi-rigid structure configured to make a snap-fit with the defibrillator's lid 55 (FIG. 16), so that electrode package 60 can be releasably secured to the defibrillator. More particularly, and looking now at FIGS. 27-29, tray 170 has a peripheral lip 190 (FIG. 27), a pair of feet 195 (FIG. 29), a pair of feet 197 (FIG. 28) and a latch 200 (FIG. 27). Correspondingly, and looking now at FIGS. 30 and 31, defibrillator lid 55 has a pair of tabs 205 (FIG. 30), a pair of feet 210 (FIG. 31) and a latch 215 (FIG. 31). Tray feet 195 (FIG. 29) engage lid tabs 205 (FIG. 30), tray feet 197 (FIG. 27) engage lid feet 210 (FIG. 31), and tray latch 200 (FIG. 27) engages lid latch 215 (FIG. 31), whereby electrode package 60 may be releasably secured to the underside of lid 55 prior to use.

In one preferred form of the invention, tray 170 is formed out of Ticona Topas 8007 material and peel-off top 175 is formed out of 48 gauge polyester/10.8# white LDPE 0.001 foil/3 mil coextrusion peel seal blend.

Although a snap-fit arrangement is used in the preferred embodiment to releasably secure electrode package 60 to lid 55, it should be appreciated that other alternative fastening arrangements may also be used.

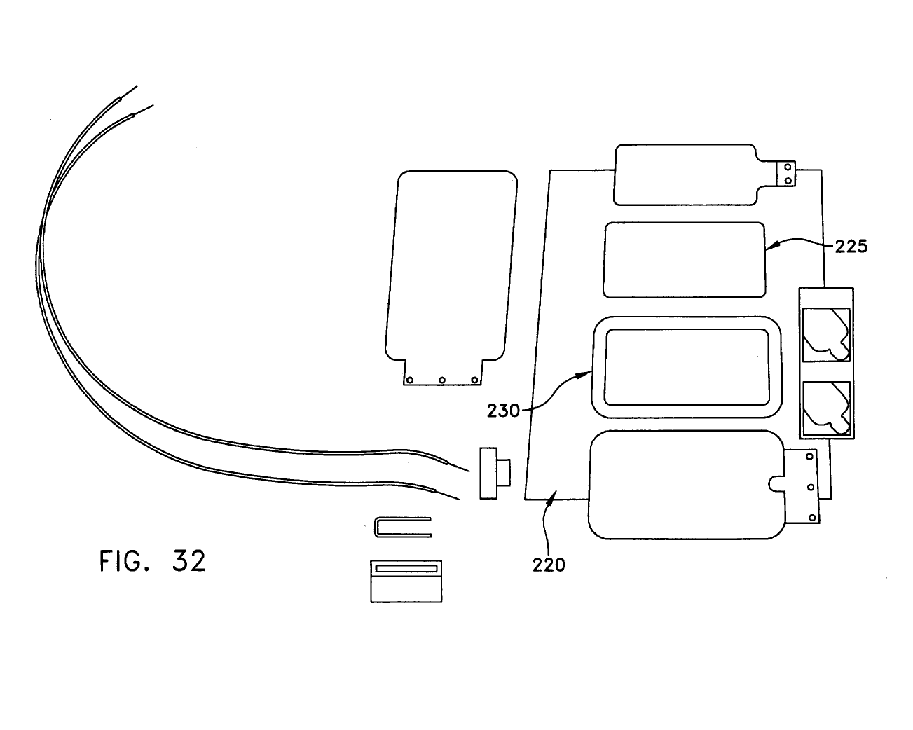

Looking next at FIGS. 32-38, electrodes 45 are preferably manufactured as follows:

1. An assembly release liner 220 is placed on a work surface (FIG. 32).

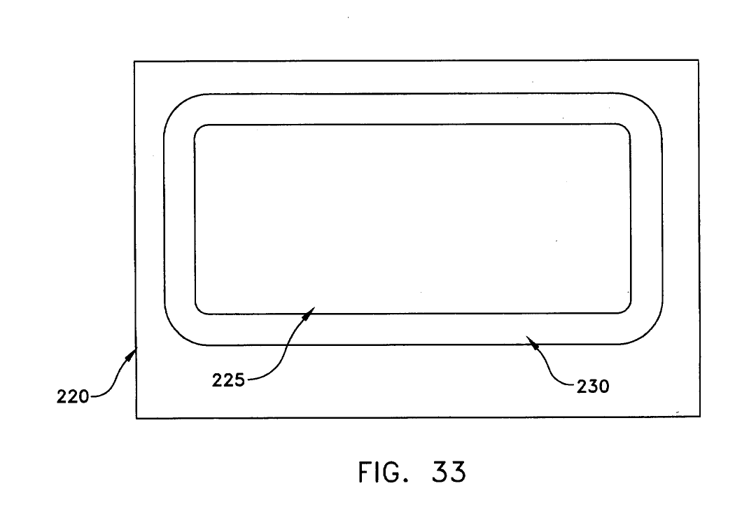

2. A hydrogel pad 225 is placed on assembly release liner 220 (FIG. 33).

3. A foam ring 230 is placed around hydrogel pad 225 (FIG. 33).

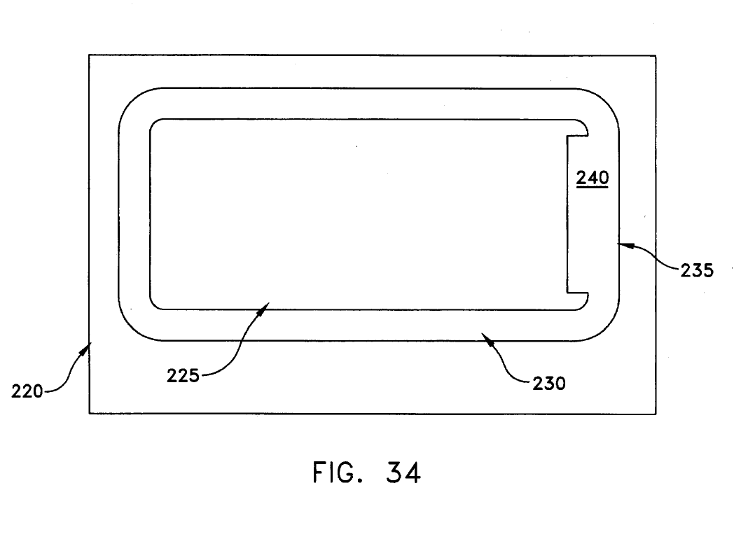

4. A double sticky tape 235, having a release liner 240 on one side thereof, is laid over a portion of hydrogel pad 225 and foam ring 230 (FIG. 34).

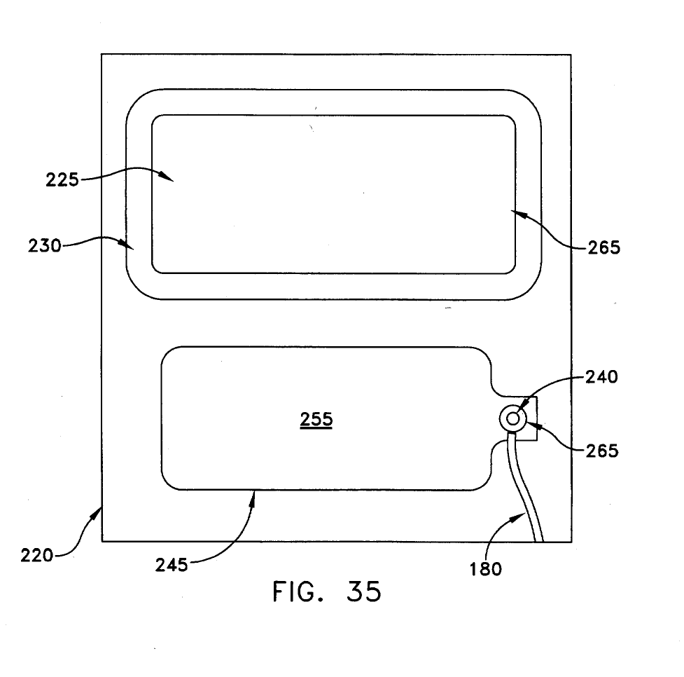

5. Release liner 240 is removed (FIG. 35).

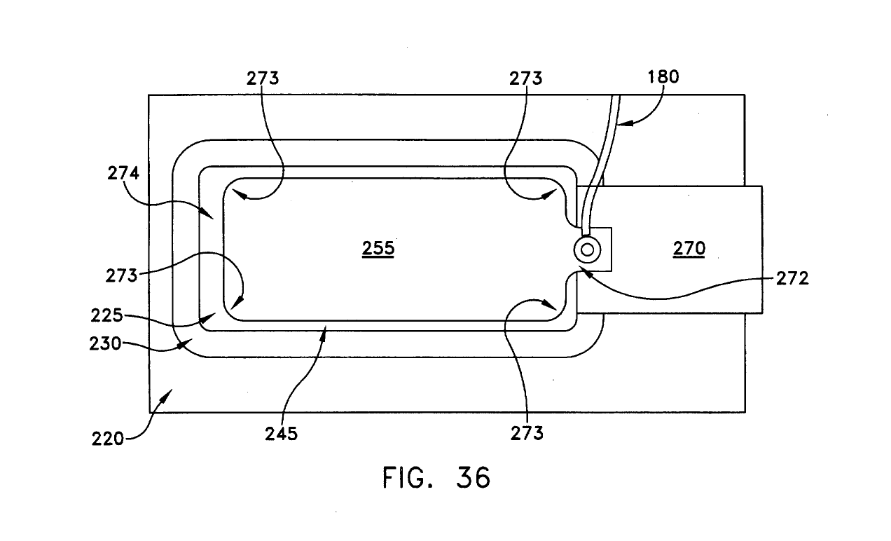

6. A conductor subassembly 245 is mounted to hydrogel pad 225 and foam ring 230. More particularly, conductor subassembly 245 is first formed by fastening a conductor lead 180 to a conductor 255 via a socket 260 and ring 265 (FIG. 35), and then subassembly 245 is mounted to hydrogel pad 225 and foam ring 230 by laying an insulator label 270 against foam ring 230 (FIG. 36), and then placing subassembly 245 against hydrogel pad 225 and foam ring 230, with socket 260 and ring 265 lying against insulator label 270. By providing the isolating double sticky tape 235 between socket 260 and the hydrogel pad 225, current is prevented from passing from socket 260 directly into the body; instead, the current is distributed throughout the substantial surface area of the complete conductor 255, whereby to enhance even electrical transmission to the patient. In this respect it should be appreciated that the presence of the insulating double sticky tape under the conductor neck 272 (FIG. 36) further promotes current distribution prior to entering the patient. In addition, the four rounded corners 273 (FIG. 36) of conductor 255 minimize current concentrations which can result in hot spots. Furthermore, conductor 255 is preferably placed against hydrogel pad 225 so as to form a slightly larger border 274 (FIG. 36) at the far end of the conductor so as to enhance current distribution at the far end of the conductor.

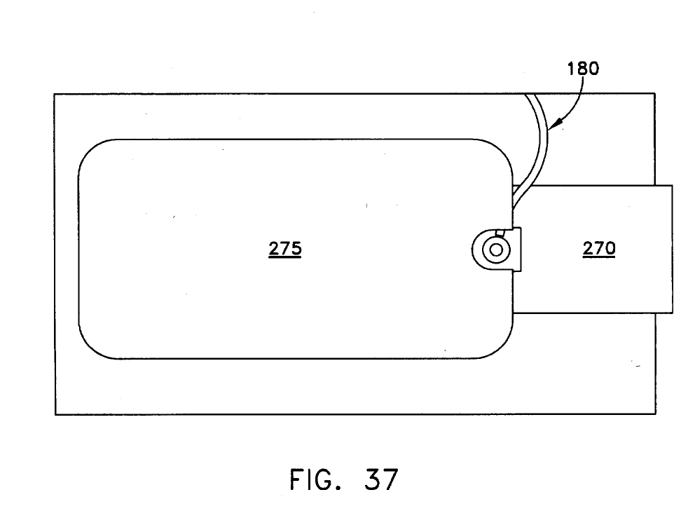

7. A foam backing 275 is placed against foam ring 230, hydrogel pad 225 and conductor 255 (FIG. 37).

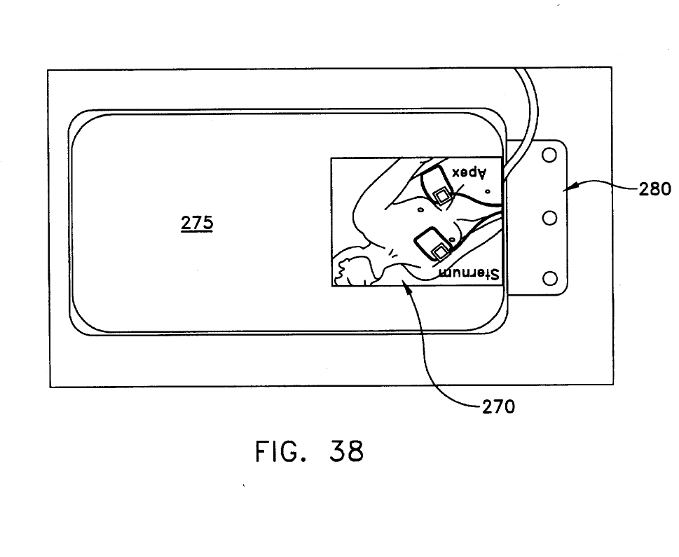

8. Insulator label 270 is folded over ring 265 (FIG. 38).

This completes assembly of one electrode.

The electrode is then lifted off assembly release liner 220 and placed against a release liner 280 (FIG. 38).

The foregoing steps 1-8 are then repeated so as to form a second electrode. Then the second electrode is mounted to the opposite side of the same release liner 280. Then release liner 280 is placed inside tray 170 and the tray is sealed with peel-off top 175 so as to form the complete electrode package 60.

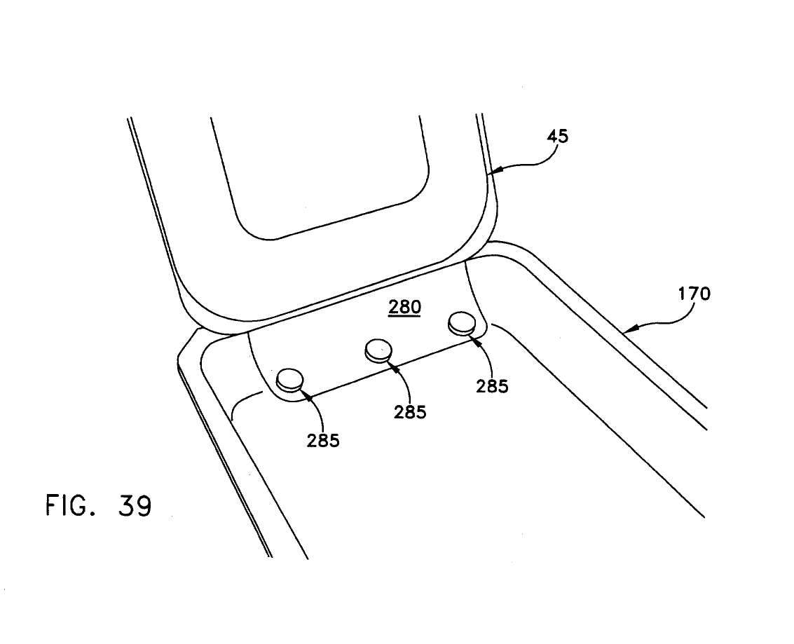

In one preferred form of the invention, and looking now at FIG. 39, release liner 280 is mounted to a plurality of posts 285 formed on tray 170, whereby the electrodes 45 will be quickly and easily presented to the user upon opening electrode package 60.

It has been discovered that the particular shape and proportions of electrode 45 provide an unusually effective mechanism for administering the desired electrical pulse to the patient: less tenting is found to occur during muscle contraction, which in turn results in less arcing, which can cause burning and loss of energy. The use of a more aggressive skin adhesive can also contribute to this effect.

Thus, with the present invention, the electrodes are placed inside a relatively rigid tray, a sheet of sealing material is used to seal the area of the tray, thereby encapsulating or sealing the electrodes within the tray, and then the entire assembly is snapped into a releasable mount on the underside of the defibrillator's lid. In use, the lid is opened, the sheet of sealing material is pulled away to expose the electrodes, and then the electrodes are applied to the patient. After a single use, the operator is able to remove the tray from the underside of the lid and dispose of it along with the used electrodes, and is able to replace the used tray with a new tray containing sealed electrodes.

And in the preferred form of the invention, the electrodes are held on opposite sides of the release liner which is mounted to the tray; opening the tray causes the two electrodes to be conveniently presented to the user, thereby facilitating handling of the electrodes and reducing the time it takes to apply the electrodes to the patient. This results in earlier first shock delivery to the patient, thus increasing their chance for survival.

### Use

Defibrillator system 35 is generally used as follows.

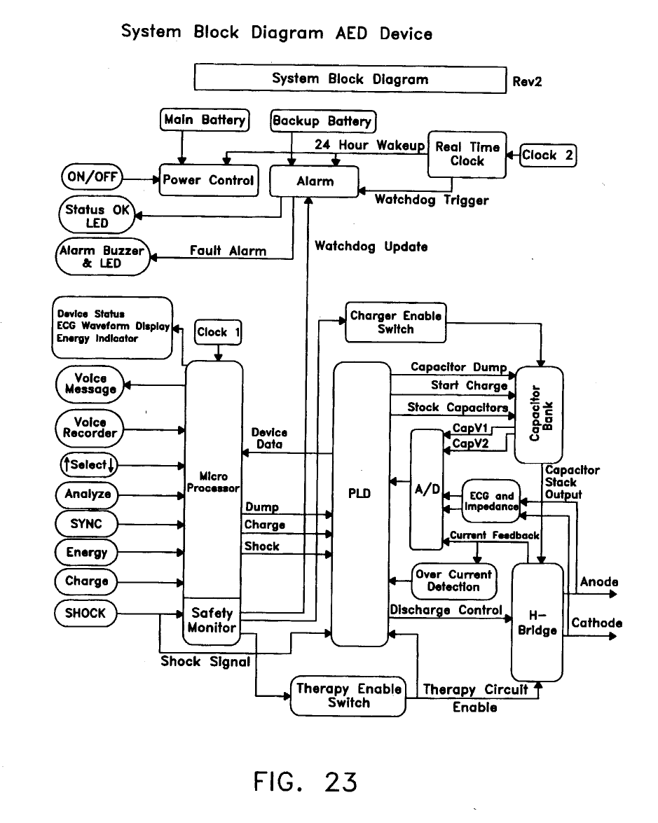

When it appears to a first-responder (e.g., a flight attendant) that a victim is in cardiac arrest, the first-responder (i.e., the user) opens lid 55 (FIG. 16) and pushes power button 95 (FIG. 17) so as to turn on the unit. Defibrillator electronics 165 (FIGS. 20 and 23) cause a voice prompt, which can be heard over speaker 105 (FIG. 20) accessed through speaker ports 110, to guide the user through the American Heart Association® (AHA) ABCD sequence of evaluating the patient's condition. Speaker 105 also allows the user to receive instructions from a remote site (e.g., a hospital) via radio link.

If it appears that defibrillation is required, the user will open the electrode package's peel-off top 175 (FIG. 26), with or without removing the electrode package from lid 55, and then connect electrode connector 185 (FIG. 26) to the defibrillator's connector slot 90 (FIG. 17), if the electrode connector 185 is not already pre-connected to the defibrillator's connector slot 90. As this occurs, the elastic nature of release liner 280 (FIG. 39) will cause the release liner to present the electrodes out of the recess of tray 170. The user then peels electrodes 45 off to release liner 280 and applies them to the chest of the patient. The defibrillator's electronics 165 (FIGS. 20 and 23) then use electrodes 45 to monitor the victim's heart to determine of defibrillation is required.

If electronics 165 determine that defibrillation is required, speaker 105 will prompt the user to depress shock button 160 (FIG. 17). When the shock button is depressed, electronics 165 cause a low voltage, low current pre-pulse (FIG. 14) to be very briefly applied to the patient, whereby the patient's thoracic impedance may be measured by electronics 165. Based on this measured impedance, electronics 165 then cause a bi-phasic shock pulse of proper peak current, tilt and duration to be applied to the patient so as to effect defibrillation. While the foregoing is occurring, microphone 115 (FIG. 20) allows sounds at the emergency site to be recorded by the unit or transmitted to a remote site (e.g., a hospital) via a radio link.

### Additional Remarks

Therefore, the present invention provides for a compact, rugged, lightweight, inexpensive, easy to use, water-resistant, and electronically efficient defibrillator that preferably uses stacked, switchable capacitors to generate a desired biphasic waveform for cardiac resuscitation. The compact defibrillator of the present invention can be broadly deployed in hospitals (or in any cardiac emergency situation) so as to provide for rapid coverage of unexpected patient events, and a greater compliance with Joint Commission on Accreditation of Healthcare Organizations (JCAHO) and other regulatory standards.

A system and method has been shown in the above embodiments for the effective implementation of an automated external defibrillator (AED) unit. While various preferred embodiments have been described and shown, it will be understood that there is no intent to limit the invention by such disclosure but, rather, it is intended to cover all modifications and alternate constructions falling within the spirit and scope of the invention. Thus, for example, while defibrillator system 35 has been described as being an automated external defibrillator (AED) system, it could also be implemented as a non-automatic external defibrillator system. Furthermore, while defibrillator system 35 has been described as comprising electrodes of the sort comprising hydrogel pads, it could also be used in conjunction with defibrillator paddles. Also, the present invention should not be limited by the mechanism via which the tray containing the electrodes is attached to the lid, and the mechanism via which the lid is attached to the body of the defibrillator unit. These and other modifications and alternate constructions are considered to fall within the spirit and scope of the present invention.

What is claimed is:

1. A defibrillator for applying a therapeutic shock pulse to a patient, said defibrillator being adapted to:
(1) measure the thoracic impedance of the patient; and
(2) provide a bi-phasic shock pulse to the patient, the bi-phasic shock pulse:
(i) being characterized by a tilt which is less than the time constant of a 100 μF capacitance; and
(ii) having a peak current limited in accordance with the measured impedance of the patient.

2. A defibrillator for applying a therapeutic shock pulse to a patient, said defibrillator being adapted to:
(1) measure the thoracic impedance of the patient; and
(2) provide a bi-phasic shock pulse to the patient, the bi-phasic shock pulse:
(i) being characterized by a tilt which is less than the time constant of a 100 μF capacitance; and
(ii) having a shock voltage selected in accordance with the measured impedance of the patient, wherein the shock voltage is generated by apparatus charged to a fixed charge voltage.

3. A defibrillator for applying a therapeutic shock pulse to a patient, said defibrillator being adapted to:
(1) measure the thoracic impedance of the patient; and
(2) provide a bi-phasic shock pulse to the patient, the bi-phasic shock pulse:
(i) being characterized by a tilt which is less than the time constant of a 100 μF capacitance;
(ii) having a peak current limited in accordance with the measured impedance of the patient; and
(iii) having a shock voltage selected in accordance with the measured impedance of the patient, wherein the shock voltage is generated by apparatus charged to a fixed charge voltage.

4. A defibrillator for applying a therapeutic shock pulse to a patient, said defibrillator being adapted to:
(1) measure the thoracic impedance of the patient; and
(2) provide a bi-phasic shock pulse to the patient, the bi-phasic shock pulse:
(i) being characterized by a tilt which varies in accordance with the measured impedance of the patient; and
(ii) being characterized by a tilt which is less than the time constant of a 100 μF capacitor.

5. A defibrillator for applying a therapeutic shock pulse to a patient, said defibrillator being adapted to:
(1) measure the thoracic impedance of the patient; and
(2) provide a bi-phasic shock pulse to the patient, the bi-phasic shock pulse:
(i) being characterized by a tilt which is less than the time constant of a 100 μF capacitance;
(ii) having a time duration selected in accordance with the measured impedance of the patient; and
(iii) having a peak current limited in accordance with the measured impedance of the patient.

6. A defibrillator for applying a therapeutic pulse to a patient, said defibrillator being adapted to:
(1) measure the thoracic impedance of the patient; and
(2) provide a bi-phasic shock pulse to the patient, the bi-phasic shock pulse:
(i) being characterized by an increased average current in accordance with the measured impedance of the patient.

7. A defibrillator according to claim 1 wherein said defibrillator uses capacitance to provide the bi-phasic shock pulse.

8. A defibrillator according to claim 1 wherein said defibrillator uses resistance to provide the bi-phasic shock pulse.

9. A defibrillator according to claim 1 wherein said defibrillator uses a stacked, switched capacitor bank to provide the bi-phasic shock pulse.

10. A defibrillator according to claim 9 wherein the measured impedance of the patient is used to determine how the capacitor bank is configured and how many of the capacitors are fired so as to provide the bi-phasic shock pulse.

11. A defibrillator according to claim 9 wherein all of the capacitors in the capacitor bank are charged to the same charge voltage.

12. A defibrillator according to claim 9 wherein said capacitor bank comprises six identical capacitors connected by three switches.

13. A defibrillator according to claim 9 wherein the thoracic impedance of the patient is measured using said capacitor bank.

14. A defibrillator according to claim 1 wherein the thoracic impedance of the patient is measured by using a pre-pulse configured to compensate for electrode-skin interactions.

15. A defibrillator according to claim 14 wherein said pre-pulse has a duration of between approximately 100 μseconds and 1 millisecond.

16. A defibrillator for applying a therapeutic shock pulse to a patient, said defibrillator comprising:
a body enclosing hardware for generating the shock pulse; and
a lid for covering all of the user accessible components of the body.

17. A defibrillator for applying a therapeutic shock pulse to a patient, said defibrillator comprising:
a body enclosing hardware for generating the shock pulse; and
a lid for covering at least a portion of said body, said lid being adapted to releasably store an electrode tray on the underside of said lid.

18. A package for storing electrodes prior to use with a defibrillator, said package comprising:
a substantially rigid tray defining a recess for receiving said electrodes; and
a peel-off sheet releasably secured to said tray so as to hermetically seal the electrodes within said recess.

19. A package according to claim 18 wherein said tray is configured for releasable attachment to the defibrillator.

20. A package according to claim 19 wherein said package further comprises a release liner for receiving the electrodes thereon, said release liner being configured and secured to said tray such that (i) said release liner will be held in said recess when said peel-off sheet is secured to said tray, and (ii) said release liner will emerge from said recess when said peel-off sheet is sufficiently detached from said tray.

21. An electrode for use in applying an electric current to a patient, said electrode comprising:
a hydrogel pad having a first generally rectangular shape with rounded corners; and
a conductor mounted to said hydrogel pad, said conductor having a second generally rectangular shape with rounded corners, with the footprint of said conductor being less that the footprint of said hydrogel pad, said conductor being configured at a first edge thereof to be connected to the circuit for applying the electric current to the patient, whereby when said conductor is mounted to said hydrogel pad, said hydrogel pad will overlap said conductor on at least the three remaining edges.

22. An electrode according to claim 21 wherein the overlap is largest at the edge opposite said first edge.

23. A defibrillator for applying a therapeutic shock pulse to a patient, said defibrillator having a footprint substantially the size of the footprint of its associated electrodes.

24. A defibrillator for applying a therapeutic shock pulse to a patient, said defibrillator comprising:
a body enclosing hardware for generating the shock pulse, said body including a communication device for accessing the hardware without opening said body.

25. A defibrillator comprising a body that encloses hardware associated with the defibrillator, and further comprising a fault analysis system comprising a visual signal indicating whether or not there is a malfunction in the hardware, said visual signal being visible without opening said body.

26. An electrode tray, where electrodes in the tray are face-to-face on a release liner.

27. An electrode connector that includes a component that allows a defibrillator to detect when the connector is inserted.

28. Electrodes in a package with an anode-to-cathode resistor, which:
(a) allow device impedance circuit testing in periodic self-tests;
(b) identify a unique electrode for shelf life duration monitoring; and
(c) the resistor can be removed or modified by the periodic self-tests by using the defibrillator pulse when shelf life has expired.

29. The defibrillator waveform:
(a) Pre-pulse detects: have thoracic impedance, detects impedance to real defibrillation currents, determines selection of capacitors, and determines waveform duration;
(b) Pre-pulse is used to determine the control of a waveform greater that 250 joules.
(c) Capacitor selection allows delivery of a pulse into thoracic impedance from 25-200 ohms without a change in capacitor voltage.
(d) Capacitor selection allows delivery of a pulse into thoracic impedance from 25-200 ohms without inserting series resistors.

30. Defibrillation waveform electronics:
(a) Patient-connected leads have leakage protection with semiconductors, from the defibrillator capacitor high voltage;
(b) Patient-connected leads are protected from a second external defibrillator (damped sine wave or multi-phasic) with semiconductors;
(c) Patient-connected leads are protected from ESD discharge with semiconductors;
(d) Double fault protection of the defibrillator high voltage to the patient leads is provided with semiconductors; and
(e) ECG monitoring allows +/−5 volt offset voltages while connected to the defibrillation high voltage capacitors.

31. Defibrillator capacitor voltage is dumped with internal discharge electronics that utilize low power, low cost semiconductors.

32. Battery for device operation is sized to perform a single patient rescue sequence and be replaced for the next patient rescue.

33. Provide fault analysis, with transmission of results on external data communications output for remote monitoring device status.

34. Capacitor charging with low voltage from battery combined with a safety dump circuit that stops dumping at a voltage just above the battery voltage.

35. A connector for detecting the nature of an associated electrode and its current use.

36. A battery for use in a defibrillator, the battery being sized for a single rescue event.

37. An automated external defibrillator comprising a key receptacle, with said defibrillator being configured for manual operation when said key receptacle is filled.

38. A defibrillator according to claim 37 wherein said key receptacle comprises a flashcard slot.

39. A defibrillator comprising a safety circuit having a shock delivery switch providing redundant control to the therapy delivery circuits.

40. A defibrillator wherein ECG monitoring and impedance monitoring utilize the same circuitry.

41. A defibrillator having an independent time base, and alarm activation, for initiating periodic self-testing.

42. A defibrillator adapted to provide continuous ECG analysis for detection of ventricular fibrillation during periods of operator contact with the patient for the purpose of expediting delivery of defibrillation shocks.

43. A defibrillator adapted to provide real-time coaching to a user during a rescue.
